(19) United States
(12) Patent Application Publication
(10) Pub. No.: US 2022/0215898 A1
(43) Pub. Date: Jul. 7, 2022

(54) METHODS AND APPARATUS FOR GENERATING A VIRTUAL MODEL OF XENOBIOTIC EXPOSURE USING TRANSCRIPTOMICS ANALYSIS OF LIQUID BIOPSY SAMPLES

(71) Applicant: Certara USA, Inc., Princeton, NJ (US)

(72) Inventors: Amin ROSTAMI-HODJEGAN, Manchester (GB); Brahim ACHOUR, Manchester (GB)

(21) Appl. No.: 17/656,302

(22) Filed: Mar. 24, 2022

Related U.S. Application Data
(63) Continuation of application No. PCT/US2020/052261, filed on Sep. 23, 2020.
(60) Provisional application No. 62/905,885, filed on Sep. 25, 2019.

(51) Int. Cl. G16B 5/00; C12Q 1/6876; G16B 25/10

(52) U.S. Cl. CPC G16B 5/00; C12Q 1/6876; C12Q 2600/106; C12Q 2600/158; G16B 25/10

(57) ABSTRACT

Processes are provided for establishing a virtual physiologically based pharmacokinetic (PBPK) model in a population comprised of a plurality of individual subjects that has been or may be exposed to a xenobiotic molecule. The processes are derived from the identification of an abundance of a protein that is involved in absorption; distribution; localization; biotransformation; and excretion of the xenobiotic molecule from a liquid biopsy of corresponding cell free RNA. Personalised PBPK models for precision dosing, as well as methods of treatment are also provided.

This application is a continuation of PCT/US2020/052261, filed Sep. 23, 2020; which claims the benefit of U.S. Provisional Application No. 62/905,885, filed Sep. 25, 2019. The contents of the above-identified applications are incorporated herein by reference in their entireties.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence Listing.txt with a creation date of Sep. 21, 2020, and a size of 810 bytes. The Sequence Listing filed via EFS-Web is part of the specification and is hereby incorporated in its entirety by reference herein.

### FIELD OF THE INVENTION

The present invention is directed towards physiologically-based pharmacokinetic (PBPK) simulation systems, methods and apparatus for the modelling of clearance and metabolism of drugs, toxins and other substances within animals, such as humans.

### BACKGROUND OF THE INVENTION

Differences in drug absorption, metabolism and clearance in any given individual may depend on a plurality of causes such as age, gender, and ethnicity to name only a few. In addition, lifestyle choices and health status can have profound effects upon a given individual's ability to absorb, distribute, metabolise or excrete pharmaceutical and other xenobiotic substances. For example, whether an individual is a smoker or suffers from a chronic disease, such as cirrhosis, will influence his/her body's ability to clear the drug. The observation of extreme side-effects or the unexpected lack of expected therapeutic effects in some individuals following administration of putatively therapeutic doses of a drug, thus, requires a fuller understanding of the issue of variability and highlights the importance of identifying covariates that determine the exposure to drugs in each individual. Although genetics may determine some variations in biological activities of organs on a drug (e.g. the genotype of an enzyme) and also of drugs on the body (pharmacodynamics), within any given genotype there are still variations which cannot be predicted with genotyping as it is carried out currently.

There exists a need to provide a more accurate way to identify safe and efficacious drug dosage for individuals in need rather than current crude population-based measures. The various factors that impact pharmacokinetics and pharmacodynamics in paediatric and geriatric contexts, for example, are quite different to those of adults (Zhou et al. Clin Pharmacol Ther. (2018) Jul;104(1):188-200). Even between adults there are considerable variations between population cohorts as a result of inter alia obesity, liver and/or renal impairment (Spanakis & Marias, (2014) In Silico Pharmacol. Dec;2(1):2). As a consequence, there is often a need to continually monitor and modify drug dose regimens in the elderly; neonates, infants, and children; as well in various adult sub-populations. Variations with a similar level of profound effect may also exist in other sub-populations and yet may be effectively disregarded by companies or clinicians when drugs are developed or prescribed.

Physiological information as well as compound-specific physicochemical data can be used to describe the complex transport processes of a chemical compound throughout the body of a human or animal subject and to simulate its in vivo performance. This model approach consists of organ and tissue compartments connected by a circuit of flowing blood, subdivided as arterial and venous blood. The properties of each compartment are described by a series of differential equations with physiological parameters to represent the system in an integrated and biologically meaningful manner and combine to form what is called a physiologically-based pharmacokinetic (PBPK) model. PBPK modellling in combination with classical population pharmacokinetic (popPK) model-based simulations is used increasingly to answer questions in drug development in order to modify dosing and dosage regimen of drugs. It is, of course, possible to determine kinetics and dynamics for a particular drug or xenobiotic in an individual and accordingly change the dose. Such an approach requires building a model for each given drug/xenobiotic in each individual as opposed to creating a virtual mirror image (Virtual Twin) of the individual which can handle any given drug or xenobiotic in the same way that the individual themselves will handle it. The construction of Virtual Twin PDPK models has been described in US Patent Application No 2016/0335412.

Building an accurate model for each individual (Virtual Twin), a priori to administration of any drug or exposure to any xenobiotic compound, requires biopsies to be taken of the most relevant tissue(s), particularly those responsible for clearance of the particular compound under study, which is often the liver. It is evident that it is impractical and potentially hazardous to expect individuals to be subjected to invasive biopsy procedures to harvest tissue from, say, the liver and kidney, simply to create an individualised model of drug metabolism and clearance. In addition, it is difficult and ethically challenging to explore complex clinical scenarios such as drug-drug interactions (DDIs) in paediatric contexts or in small sub-populations having rare genetic variations of drug metabolizing enzymes. As a result of such complexity and inconvenience there has been little effort in the field to pursue models that can provide accurate predictions of drug absorption, distribution, clearance and metabolism at the individual level. Hence, there exists a barrier to the creation of PBPK models that allow for the adoption of personalised point of care dosage regimens for drugs. Consequently, the problem of over- and under-dosing, as well as failure to predict adverse DDIs, is perpetuated.

A survey of the contribution of specific enzyme families to drug metabolism showed that cytochrome P450 and glucuronosyltransferase enzymes mediate the direct metabolism of approximately 90% of the 200 most prescribed drugs in the US in 2002 (Wienkers, L. C. & Heath, T. G. Nature reviews. Drug discovery 4, 825-833 (2005)). This trend has changed very little in recent years. The drug-eliminating function of the liver is also predicated on drug transporters, which play a key role in trafficking drugs and their metabolites in and out of liver cells. The interplay between the function of enzymes and transporters therefore dictates a patient's exposure to a certain drug. Guiding patient stratification and individual dosing, therefore, crucially requires quantitative characterization, beyond genetics, of enzymes and transporters in individual patients that are involved in metabolism, distribution, excretion and transport of xenobiotics.

Recent advances in ‘omics’ methods have led to improved characterization of clinically relevant proteins in human tissue samples, with applications ranging from disease diagnostics to monitoring of therapy. However, the use of tissue biopsies is not feasible for routine clinical practice with the objective of characterizing individual metabolic capacity. Hence, research has so far relied on opportunistic samples, which are often of sub-optimal quality. Therefore, the development and validation of an effective and less invasive test is highly desirable, especially for drug dose individualization, which should afford a substantial advance in the area of precision dosing.

Cell free nucleic acids are present in the bloodstream and include RNA, so-called ‘circulating RNA’, despite the typically very short half-life of RNA outside cells (EI-Hefnawy et al., Clin Chem, 2004). RNA molecules of this nature therefore are indicated to be associated with lipids, such as vesicles and lipoproteins, to enable their survival. Circulating RNA includes mRNA, which can be enriched in microvesicles or exosomes released by cells.

WO-A-02/00935 (Ramanathan) provides a description of a method for estimating the levels of certain drug metabolizing enzymes in liver—so-called “drug clearance markers”—by correlating to levels of mRNAs found in the blood cells of an individual. In Ramanathan, mRNA is isolated from a blood sample, and reverse transcribed to form cDNA, which is then analysed on a DNA microarray in order to estimate the presence and amount of protein levels of drug clearance markers in the liver based on the corresponding levels of hepatic mRNA expression. There are problems in the methodology of Ramanathan because it relies upon two assumptions:
1) that there is a direct correlation between the levels of mRNA in the blood with corresponding levels of mRNA for a given enzyme or transporter in the liver of that individual; and
2) that the individual liver mRNA levels correspond in a linear fashion to the amount of protein of the same liver enzymes and transporter present in that individual.

Ramanathan's own experiments rely on correlations between tested levels of mRNAs of different enzymes in blood samples from a first group of individuals with previously reported prior art measures of corresponding liver enzymes from a second group of different individuals. Hence, no meaningful correlations can be determined from the Ramanathan studies for any given specific enzyme and transporter as the alleged correlation occurs for a set of different enzymes and transporters between samples taken from blood and liver of different individuals. Indeed, many factors affect the translation of mRNAs into a given protein and that for any one given gene expression product there may be multiple regulatory mechanisms that control its translation into a corresponding functional protein. In the case of human-derived hepatocytes, for a several thousand-fold increase in mRNA, there might be only a few-fold change in the actual level of protein (as reviewed by Einolf et al, Clin Pharmacol Ther. 2014 Feb;95(2):179-88). Such effects may also be highly susceptible to environmental, genetic and lifestyle factors that can modulate the level and activities of drug clearance enzymes in vivo on an individual basis.

A further complication arises from a phenomenon described as “shedding” of mRNA by the cells of an organ or tissue often within exosomes into bodily fluids, such as into the bloodstream. The amount of shedding varies between individuals with “fast shedders” releasing a higher amount of RNA for the same amount of transcription of a particular gene in the originating organ or tissue when compared to that released by “slow shedders”. It can be appreciated, therefore, that quantification of circulating RNA alone without correction for the level of shedding within an individual will be only of limited use in accurately predicting the protein levels derived from expression of a particular gene in organ tissue. Hence, assertions in the art that circulating mRNA, of exosomal or other origin, may serve as a source of “liquid biopsy” for correlation with abundance of organ drug handling proteins are at best speculative and at worst highly premature in addressing the significant technical problems that exist.

International Patent Application No. PCT/US2019/24379 provides a liquid biopsy technology platform for transcriptomics analysis of a sample of bodily fluid taken from a human or animal subject (e.g. blood) to determine the concentration of one or more target mRNAs in the sample. The concentration of the target mRNA is adjusted with a shedding correction factor that compensates for an individual subject's level of mRNA shedding for the organ/tissue source of the target mRNA. The concentration of the one or more target mRNAs in the liquid biopsy sample may be correlated to protein abundance and, thus, functional activity in the source organ/tissue.

Efforts to develop liquid biopsy approaches further, have been limited to specific contexts such as that described in Rowland et al. (Br J Clin Pharmacol. 2019 Jan;85(1):216-226). In this study, the authors sought to demonstrate the presence of CYP3A4 and UDP-glucuronosyltransferase (UGT) proteins and mRNAs in isolated human plasma exosomes and evaluate the capacity for exosome-derived biomarkers to characterize variability in CYP3A4 activity in patients treated with midazolam. This study focused on a probe activity assay of only CYP3A4 against midazolam in a small number of healthy subjects (n=6) and demonstrated the usefulness of their approach only under induced conditions for the target enzyme within the same cohort. Most importantly, the study crucially lacked measurement of the abundance of the relevant proteins in matching liver tissue samples. These limitations restrict the applicability of the described approaches to develop robust models covering multiple drugs because they rely upon an indirect translation of midazolam activity back to hypothetical literature-inferred tissue abundance of CYP3A4 before calculating clearance values only for those drugs metabolized by CYP3A4. Such inference comes with a plethora of uncertainties related to, for example, blood flow and/or protein binding in each individual during deconvolution. Moreover, Rowland et al. did not establish correlations between the specific probe activity and liquid biopsy expression at baseline (prior to induction by rifampicin).

Hence, there remains a need to provide a practical means for generating and expanding the number of accurate and quantitative PBPK models for individuals, populations and sub-populations. There also exists a need to identify and stratify particular novel subpopulations for whom PBPK modelling would be of substantial benefit in the design of clinical trials and the administration of precision dosing.

These and other uses, features and advantages of the invention should be apparent to those skilled in the art from the teachings provided herein.

### SUMMARY OF THE INVENTION

Accordingly, a first aspect of the invention provides a process for establishing a virtual physiologically based pharmacokinetic (PBPK) model in a population comprised of a plurality of individual subjects that has been or may be exposed to a xenobiotic molecule, the process comprising the steps of:
a) isolating total cell free RNA (cfRNA_{TOTAL}) from a liquid biopsy obtained from each individual subject comprised within the population;
b) quantifying an amount of a first cell free RNA (cfRNA) present in the liquid biopsy, wherein the first cfRNA originates from a specified organ/tissue within the bodies of the subjects, and wherein the first cfRNA encodes a protein from the organ/tissue that is involved in pharmacokinetic activity relevant to the xenobiotic molecule selected from one or more of the group consisting of: absorption; distribution; localization; biotransformation; and excretion of the xenobiotic molecule;
c) performing an adjustment function on the amount of the first cfRNA so as to correct for inherent levels of RNA shedding within each of the plurality of individual subjects;
d) identifying the abundance of the protein within the specified organ/tissue for each subject by comparison of the corrected amount of the first cfRNA with abundance data for the corresponding amount of protein in the specified organ/tissue;
e) determining a pharmacokinetic activity relevant to the xenobiotic molecule for each individual subject based upon the abundance of the protein within the specified organ/tissue of the subject;
f) combining the pharmacokinetic activities of each individual subject to create a data set of pharmacokinetic activities for the population of individuals; and
g) utilising the data set to generate the PBPK model.

In a second aspect, the invention provides a process for establishing a personalised PBPK model for an individual subject that has been or may be exposed to a xenobiotic molecule, the process comprising the steps of:
i isolating total cell free RNA (cfRNA_{TOTAL}) from a liquid biopsy obtained from the individual subject;
ii quantifying an amount of a first cell free RNA (cfRNA) present in the liquid biopsy, wherein the first cfRNA originates from a specified organ/tissue within the body of the subject that is involved in pharmacokinetic activity relevant to the xenobiotic molecule selected from one or more of the group consisting of: absorption; distribution; localization; biotransformation; and excretion of the xenobiotic molecule;
iii performing an adjustment function on the amount of the first cfRNA so as to correct for inherent levels of RNA shedding in the individual subject;
iv identifying the abundance of the protein within the organ/tissue of the subject by comparison of the corrected amount of the first cfRNA with abundance data for the corresponding amount of protein in the organ/tissue;
v determining a pharmacokinetic activity relevant to the xenobiotic molecule for the individual subject based upon the abundance of the protein within the organ/tissue of the subject; and
vi generating the virtual PBPK model for the individual subject.

A third aspect of the invention provides for a method of treating an individual subject, wherein the individual is the intended recipient of a pharmaceutical treatment, the method comprising establishing a personalised virtual PBPK model in the body of the individual subject prior to or during treatment, the process comprising the steps of:
isolating total cell free RNA (cfRNA_{TOTAL}) from a liquid biopsy obtained from the individual subject;
quantifying an amount of a first cell free RNA (cfRNA) present in the liquid biopsy, wherein the first cfRNA originates from a specified organ/tissue within the body of the subject, and wherein the first cfRNA encodes a protein from the organ/tissue that is involved in pharmacokinetic activity relevant to the pharmaceutical compound selected from one or more of the group consisting of: absorption; distribution; localization; biotransformation; and excretion of the pharmaceutical compound;
performing an adjustment function on the amount of the first cfRNA so as to correct for inherent levels of RNA shedding in the individual subject;
identifying the abundance of the protein within the organ/tissue of the subject by comparison of the corrected amount of the first cfRNA with abundance data for the corresponding amount of protein in the specified organ/tissue of the subject;
determining a pharmacokinetic activity relevant to the pharmaceutical compound for the individual subject based upon the abundance of the protein within the specified organ/tissue of the subject;
generating the personalised virtual PBPK model of pharmaceutical compound clearance for the individual subject; and
treating the individual according to a dosage regimen for the pharmaceutical compound that is optimized to the individual based upon their personalised virtual PBPK model.

In embodiments of the invention the adjustment function comprises identifying the amount of the first cfRNA present by correcting against a RNA organ Shedding Correction Factor (SCF) that is determined for the individual subject by:
performing an analysis of the cfRNA_{TOTAL} in order to quantify an amount of mRNA present within the cfRNA_{TOTAL} that corresponds to each of two or more marker genes, wherein a marker gene is defined as a gene that is expressed principally and consistently in the organ/tissue; and
determining SCF as the mean concentration of mRNA of each of two or more marker genes present within the cfRNA_{TOTAL}.

In one embodiment of the invention, the SCF is determined for the subject by isolating cfRNA_{TOTAL} from a liquid biopsy obtained from an individual subject, performing an analysis of the cfRNA_{TOTAL} in order to quantify an amount of two or more marker genes mRNAs present, designated as [cfRNA]_{Marker}, wherein a marker gene is defined as a gene that is expressed principally and consistently in the organ/tissue and at a relatively high level within the dynamic range of expression specific to the organ/tissue; and determining the SCF according to the formula A:

SCF=Σᵢ₌₁^{N}[cfRNA]_{Marker}_{ᵢ}/(N×[cfRNA]_{TOTAL}) A

where N is equal to the number of marker genes quantified.

Suitably, at least three, suitably at least five, typically at least eight and optionally at least ten or more marker genes are selected in order to determine the SCF. Optionally, the organ is selected from one or more of the group consisting of: the liver; the kidney; the gut (e.g. G.I. tract); the brain/CNS; and the pancreas. In a specific embodiment the organ is the liver.

Where the organ/tissue is or comprises the liver, then at least one of the two or more marker genes may be selected from the group consisting of: A1BG (Alpha-1 -B glycoprotein); AHSG (alpha-2-HS-glycoprotein); ALB (Albumin); APOA2 (Apolipoprotein A-II); C9 (Complement component 9); CFHR2 (Complement factor H-related 5); F2 (Coagulation factor II (thrombin)); F9 (Coagulation factor IX); HPX (Hemopexin); SPP2 (Secreted phosphoprotein 2); TF (Transferrin); MBL2 (mannose-binding lectin (protein C) 2); SERPINC1 (Serpin peptidase inhibitor, clade C (antithrombin), member 1); and FGB (Fibrinogen beta chain).

Where the organ/tissue is or comprises the gut the at least one of the two or more marker genes may be selected from the group consisting of: FABP6 (fatty acid binding protein 6); VIL1 (villin 1); LCT (lactase); DEFA6 (defensin alpha 6); DEFA5 (defensin alpha 5); CCL25 (C-C motif chemokine ligand 25); RBP2 (retinol binding protein 2); APOA4 (apolipoprotein A4); REG3A (regenerating family member 3 alpha); FABP6 (fatty acid binding protein 6); MEP1B (meprin A subunit beta); ALPI (alkaline phosphatase, intestinal); and CPO (carboxypeptidase O).

Where the organ/tissue is or comprises the brain/CNS at least one of the two or more marker genes may be selected from the group consisting of: OPALIN (oligodendrocytic myelin paranodal and inner loop protein); GFAP (glial fibrillary acidic protein); OMG (oligodendrocyte myelin glycoprotein); OLIG1/2 (oligodendrocyte transcription factor 1/2); GRIN1 (glutamate ionotropic receptor NMDA type subunit 1); NEUROD6 (neuronal differentiation 6); CREG2 (cellular repressor of E1A stimulated genes 2); NEUROD2 (neuronal differentiation 2); ZDHHC22 (zinc finger DHHC-type containing 22; KCNJ9 (potassium voltage-gated channel subfamily J member 9); GPM6A (glycoprotein M6A); PLP1 (proteolipid protein 1); and MBP (myelin basic protein).

Where the organ/tissue is or comprises the kidney at least one of the two or more marker genes may be selected from the group consisting of: UMOD (uromodulin); KCNJ1 (potassium voltage-gated channel subfamily J member 1); TMEM174 (transmembrane protein 174); NPHS2 (podocin); AQP2 (aquaporin 2); TMEM52B (transmembrane protein 52B); CTXN3 (Cortexin 3); TMEM27 (transmembrane protein 27); SOST (sclerostin); and CALB1 (Calbindin 1).

Typically, the first cfRNA encodes an organ protein. Suitably, the organ-derived cfRNA encodes a xenobiotic handling protein that possesses a pharmacokinetic function or activity selected from the group consisting of: a xenobiotic clearance protein; a xenobiotic metabolising enzyme; and a xenobiotic transporting protein.

In embodiments of the invention, the xenobiotic is a pharmaceutical compound or drug.

Optionally, the first cfRNA encodes an enzyme. In one embodiment of the invention, the enzyme comprises a cytochrome P450 monooxygenase (CYP) protein. Suitably, the CYP is selected from one of the group consisting of: CYP1A1; CYP1A2; CYP1B1; CYP2A6; CYP2A7; CYP2A13; CYP2B6; CYP2C8; CYP2C9; CYP2C18; CYP2C19; CYP2D6; CYP2E1; CYP3A4; CYP3A5; and CYP3A7. In a further embodiment, the enzyme comprises a transferase selected from one of the group consisting of: a methyltransferase; a sulfotransferase; an N-acetyltransferase; a glucuronosyltransferase including, but not limited to, one or more of the group consisting of UGT1A1, UGT1A3, UGT1A4, UGT1A6, UGT1A9, UGT2B4, UGT2B7, UGT2B15 and UGT2B17; a glutathione-S-transferase; and a choline acetyl transferase.

In another embodiment, the transport protein is an ATP-binding cassette (ABC) transporter or a solute carrier (SLC) transporter.

According to embodiments of the invention, the liquid biopsy comprises a sample of a bodily fluid selected from one of the group consisting of: blood; urine; saliva; semen; tears; lymphatic fluid; bile; cerebrospinal fluid; ascites; pleural effusion; stool; and a mucus secretion. In embodiments where the liquid biopsy comprises blood or a component thereof, it may comprise whole blood, serum and/or plasma.

In further embodiments of the invention the methods described provide for quantifying the amount of at least a second cell free RNA (cfRNA), or a third, fourth, fifth, sixth, seventh or more cfRNAs present in the liquid biopsy. In a specific embodiment, a plurality of cfRNAs are quantified each one of the pluralities of cfRNAs corresponding to a different organ/tissue protein as defined herein.

According to embodiments of the invention determination of the clearance capacity for the individual subject based upon the abundance of the protein within the organ/tissue of the subject is achieved by use of an abundance curve or pre-determined function.

It will be appreciated that the features of the invention may be subjected to further combinations not explicitly recited above.

### DRAWINGS

The invention is further illustrated by reference to the accompanying drawings in which:

FIG. 1 shows an illustration of mRNA shedding from an organ, in this instance the liver is shown as the tissue of origin.

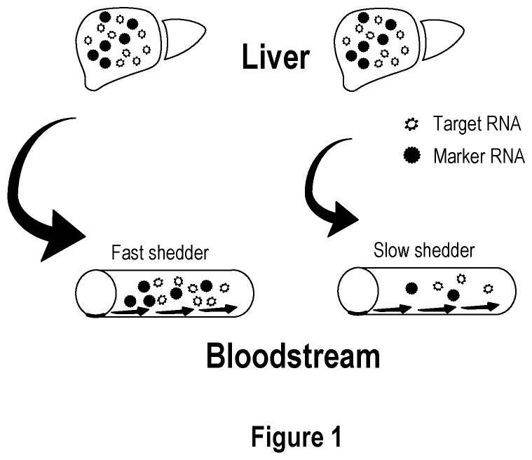

FIG. 2 shows a diagram setting out the design of a clinical trial to inform and develop an improved PBPK model for drug clearance for a chosen cohort of subjects.

FIG. 3 shows the results of in silico drug trials with liquid biopsy technology according to an embodiment of the invention for three drugs: alprazolam, midazolam and ibrutinib following uniform, stratified or individualized dosing, (a) is a bar chart showing stratified dosing relied on a dose ratio for each of three groups of patients (top quartile, bottom quartile and middle 50%) relative to a uniform dose from left to right of alprazolam (0.5 mg), midazolam (5 mg) and ibrutinib (140 mg), (b) shows a graph in which individualized dosing was performed based on individual dosage adjustment using patient-specific ratios relative to the defined uniform dose given the value of 1, (c) is a graph showing simulation of the level of drug exposure (area under the curve (AUC) of the plasma concentration-time profile) after an oral dose of the three drugs; similar levels of the drug reached the systemic circulation over time in the three dosing scenarios, the whiskers represent the range of AUC, boxes represent the 25th and 75th centiles, the lines are the medians and the + signs are the means, (d) is a line graph showing the reduction in variability in exposure following stratified and individualized dosing informed by liquid biopsy was observed in all cases.

### DETAILED DESCRIPTION OF THE INVENTION

Unless otherwise indicated, the practice of the present invention employs techniques of chemistry, computer science, statistics, molecular biology, microbiology, recombinant DNA technology, and chemical methods, which are within the comprehension of a person of ordinary skill in the art. Such techniques are also explained in the literature, for example, T. Cormen, C. Leiserson, R. Rivest, 2009, Introduction to Algorithms, 3rd Edition, The MIT Press, Cambridge, Mass.; L. Eriksson, E. Johansson, N. Kettaneh-Wold, J. Trygg, C. Wikstom, S. Wold, Multi- and Megavariate Data Analysis, Part 1, 2nd Edition, 2006, UMetrics, UMetrics AB, Sweden; M. R. Green, J. Sambrook, 2012, Molecular Cloning: A Laboratory Manual, Fourth Edition, Books 1-3, Cold Spring Harbor Laboratory Press, Cold Spring Harbor, N.Y.; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

An embodiment of the present invention provides a modelling and simulation based system, which integrates transcriptomics (e.g. RNAomics) information from liquid biopsy with in silico, in vitro, and in vivo preclinical data from a wide range of sources with mechanism-based models to anticipate and predict the exposure and effects of xenobiotic molecules in humans or animals. The model utilizes empirical and descriptive algorithms to describe the linkage between drug—or other xenobiotic—concentration, together with an observed response in various body tissues on drug clearance, especially in organs such as the liver, kidney, brain/CNS or gut. In addition, the methods utilise absorption, distribution, metabolism and excretion (ADME) databases for ‘bottom-up’ mechanistic modelling and simulation of the processes of oral absorption, tissue distribution, metabolism and excretion of drugs and drug candidates in healthy and diseased individuals as well as in populations.

Prior to setting forth the invention, definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may include DNA and RNA, subtypes of these such as genomic DNA, mRNA, miRNA, tRNA and rRNA. In embodiments of the invention mRNA is isolated from a liquid biopsy sample. Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers or probes for use in manipulation or detection of DNA such as via polymerase chain reaction (PCR).

The term “amino acid” in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties that are characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

A “polypeptide” is a polymer of amino acid residues joined by peptide bonds, whether produced naturally or in vitro by synthetic means. Polypeptides of less than around 12 amino acid residues in length are typically referred to as “peptides” and those between about 12 and about 30 amino acid residues in length may be referred to as “oligopeptides”. The term “polypeptide” as used herein denotes the product of a naturally occurring polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term “protein” is used herein to refer to a macromolecule comprising one or more polypeptide chains.

As used herein the term “biomarkers” may comprise cells, cellular components, peptides, polypeptides, proteins, ncRNA, genomic DNA, metabolites, cytokines, antigens, and polysaccharides; as well as physiological parameters such as cell count, temperature, O₂ level, CO₂ level, or pH. Biomarkers may also comprise mRNA coding for polypeptides that are not involved in xenobiotic clearance. Suitably, the biomarkers comprise a combination of features.

The term “levels” is used herein to define terms of quantity or abundance of a specified factor and may be defined in molar or absolute amounts (i.e. micrograms or milligrams etc.), concentration (e.g. mg ml⁻¹ or mol g⁻¹ etc.), and/or in terms of a specific activity (e.g. units of activity in a standard assay). The selected “level” will be appreciated as appropriate to a given factor, for example, where it is appropriate to define the amount of a given enzymatic factor by its specific activity, it may be that this measure is selected rather than the actual amount (in mg/ml) of that factor that may be present. The term “normal level”, when in the context of levels of gene or polypeptide expression, is used herein to denote the level of gene expression or enzymic activity in healthy non-diseased organs, tissue or samples. Normal levels of expression or activity represent the baseline or control level of expression of a gene. Aberrant levels in cells, either at levels that exceed the normal range or that are too low, are considered not to be normal and can be indicative of disease in the samples from which the cells have been obtained, e.g. cancer. The term “high” in relation to levels may refer to strong, consistent and/or readily detectable expression and may not be considered as necessarily aberrant.

The term “allelic variant” is used herein to denote any two or more alternative forms of a gene occupying the same chromosomal locus and controlling the same inherited characteristic. Allelic variation arises naturally though mutation, and may result in phenotypic polymorphism within populations. Gene mutations typically result in an altered nucleic acid sequence and in some cases an altered polypeptide sequence also. As used herein, the term “allelic variant” is additionally used to refer to the protein or polypeptide encoded by the allelic variant of a gene.

The term “isolated”, when applied to a polynucleotide sequence, denotes that the sequence has been removed from its natural organism of origin and is, thus, free of extraneous or unwanted coding or regulatory sequences. The isolated sequence is suitable for use in recombinant DNA processes and within genetically engineered protein synthesis systems. Such isolated sequences include cDNAs and genomic clones. The isolated sequences may be limited to a protein encoding sequence only (e.g. an mRNA), or can also include 5′ and 3′ regulatory sequences such as promoters, transcriptional terminators and UTRs.

The term “isolated”, when applied to a polypeptide is a polypeptide that has been removed from its natural organism of origin. Typically, the isolated polypeptide is substantially free of other polypeptides native to the proteome of the originating organism. Suitably, the isolated polypeptide may be in a form that is at least 95% pure, more suitably greater than 99% pure. In the present context, the term “isolated” is intended to include the same polypeptide in alternative physical forms whether it is in the native form, denatured form, dimeric/multimeric, glycosylated, crystallised, or in derivatized forms.

As used herein, the term “organ” is synonymous with an “organ system” and refers to a combination of tissues and/or cell types that may be compartmentalised within the body of a subject to provide a biological function, such as a physiological, anatomical, homeostatic or endocrine function. Suitably, organs or organ systems may mean a vascularized internal organ, such as a liver, kidney, brain, gut or pancreas; or may comprise fluid organ systems such as the blood and circulatory system. Typically organs comprise at least two tissue types, and/or a plurality of cell types that exhibit a phenotype characteristic of the organ. In contrast “tissue” refers to an aggregation or population of cells of the same or a similar type and/or lineage that may cooperate with other tissues to form an organ system.

The term “sample” is used to describe isolated materials of biological origin that can be used for a diagnostic, analytical or prognostic purpose. Biological materials may be analysed in tissue microarrays, or via other assay methods, and can include tissues from specific organs such as liver, kidney, brain, heart, epithelium, lung, and bone, as well as other tissues; as well as fluid materials such as whole blood, plasma, serum, lymph, urine, stool, cerebrospinal fluid, ascites, pleural effusion and saliva etc. Such materials may also include in vivo and in vitro cellular materials such as healthy or diseased cells, tissues and cell lines—e.g. cancer cell lines, which may be manipulated for in vitro purposes—e.g. immortalised cell lines or induced pluripotent stem cells. The macromolecules analysed in these materials typically include polypeptides such as proteins as well as polynucleotides such as RNA (including mRNA), and DNA.

The term “blood sample” may refer to any or all of whole blood, plasma, serum, erythrocyte and/or leucocyte fractions, and any other blood derivative. Blood samples may be comprised within a liquid biopsy obtained from an individual or plurality of individuals.

The term “microsome” refers to vesicles made by re-forming of the endoplasmic reticulum (ER) during the break-up of cells in vitro, which can be concentrated and isolated from other cell debris. Cytochrome P450 monooxygenase enzymes (CYPs) are present in ER and so microsomal preparations containing CYPs can be obtained from tissue samples such as organ tissue (e.g. liver), where CYPs are highly abundant. CYPs are further discussed below.

The term “microvesicle” or “exosome” relates to extracellular vesicles that may be produced or shed by cells for example by exocytosis, budding or blebbing of the plasma membrane. Cell death by apoptosis may also lead to microvesicle production. Microvesicles are found in interstitial space and in many body fluids, and may contain mRNA, miRNA and/or proteins. It is thought that methods of intercellular communication may rely on microvesicle transport. Exosomes are a type of microvesicle that range in size from nanometer scale through to micrometer size. Exosomes are derived from parental cells comprised within organs or tissues so they are able to reflect both the physiological and pathophysiological state of those parental cells.

“Cell free nucleic acid” may be DNA, RNA, or any combination thereof. The nucleic acid may be cell free DNA (cfDNA), cell free RNA (cfRNA), or any combination thereof. The samples from which the cell free nucleic acids may be isolated include any bodily fluid capable of providing a liquid biopsy. Where the liquid biopsy comprises blood, the cell free nucleic acids may be located within plasma or serum.

As used herein, the phrases “xenobiotic or drug metabolizing enzymes” or “xenobiotic or drug clearance proteins” will include cytochrome P450 monooxygenase enzymes (CYPs) as well as membrane transport proteins, and transferases. In embodiments of the invention the CYP enzymes are selected from human CYP families 1,2 and 3, which are the CYP families typically linked to xenobiotic (e.g. drug) metabolism and clearance. Suitably the CYPs may comprise any, some or all of the CYPs selected from the group consisting of: CYP1A1; CYP1A2; CYP1B1; CYP2A6; CYP2A7, CYP2A13; CYP2B6; CYP2C8; CYP2C9; CYP2C18; CYP2C19; CYP2D6; CYP2E1; CYP3A4; CYP3A5 and CYP3A7. CYPs are haemoproteins, that is, of the superfamily of proteins containing haem (or heme) as a cofactor. These proteins are involved in the metabolism of xenobiotics, in general by oxidation reactions involving NADPH and oxygen. Different drugs often have different CYP proteins involved in their metabolism, a selection of exemplary compounds that are substrates for corresponding metabolizing CYPs are listed below—it will be appreciated that this list is non-exhaustive.

| | |
|---|---|
| CYP1 A2 | Caffeine; Tacrine; Theophylline; Melatonin; |
| | Clozapine; Lidocaine |
| CYP2A6 | Bilirubin; Cortinine; Coumarin |
| CYP2B6 | Benzphetamine; Buproprion; |
| | Methamphetamine; Temazepam; |
| CYP2C8 | Amodiaquine; Paclitaxel; Ibuprofen |
| CYP2C9 | Diclofenac; Irbesartan; Valsartan; Ibuprofen; |
| | Tamoxifen; Tolbutamide |
| CYP2C19 | Hexobarbital; Imipramine; Melatonin; |
| | Omeprazole; Diazepam |
| CYP2D6 | Codeine; Dihydrocodeine; Amphetamine; |
| | Loratidine; Oxycodone; Paroxetine; |
| | Risperidone; Tamoxifen |
| CYP2E1 | Aniline; Chlorzoxasone; Halothane; |
| | Isoflurane; para-Nitrophenol; Vinylchloride |
| CYP3A4/5 | Alfentanil, Alprazolam; Atorvastin; Cortisol; |
| | Cholesterol; Dasatinib; Dexamethasone; |
| | Diazepam; Midazolam; Prednisolone; |
| | Quinine; Sildenafil; Testosterone; |
| | Triazolam; Vincristine |

(Zanger & Schwab (2013) Pharmacology & Therapeutics 138 (2013) 103-141; Watari et al. (2019) Biol. Pharm. Bull. 42, 348-353);

The CYP3A subclass catalyzes an extensive number of oxidation reactions of clinically important drugs as shown above. It is currently believed that greater than 60% of clinically used drugs are metabolized by the CYP3A4 enzyme, including several major drug classes. Hence, accurately determining the abundance of even CYP3A4 alone in the liver of an individual subject based upon a liquid biopsy would facilitate the development of virtual PBPK models that would be able to predict that individual's capacity for clearance of dozens of approved drugs currently on the market.

Other, non-CYP, proteins that are involved in metabolism of xenobiotic molecules include transferases: enzymes that catalyse the transfer of a functional group from a donor molecule to a specified substrate molecule (an acceptor) which is typically a drug or other xenobiotic compound. Transferase enzymes involved in drug metabolism are typically those that catalyse conjugation of moieties such as glutathione, methyl groups, acetyl groups, sulfate, and amino acids to a substrate molecule which may include a drug or a metabolite of a drug. Exemplary drug metabolizing transferases may include methyltransferases; sulfotransferases; N-acetyltransferases; glucuronosyltransferases (UDP-glucuronosyltransferases or UGTs) including, but not limited to, one or more of the group consisting of UGT1A1, UGT1A3, UGT1A4, UGT1A6, UGT1A9, UGT2B4, UGT2B7, UGT2B15 and UGT2B17; glutathione-S-transferases; and choline acetyl transferases.

In addition to the above, membrane bound and non-membrane bound transport proteins also may influence the levels of xenobiotic (or metabolite) compound uptake and, hence, the levels of metabolism and clearance of a given compound within the body of an individual. Transport proteins may include one or more of the group selected from: transmembrane pumps, transporter proteins, escort proteins, acid transport proteins, cation transport proteins, vesicular transport proteins and anion transport proteins. Exemplary transporter proteins include ATP-binding cassette (ABC) transporters including, but not limited to, one or more of the group selected from: ABCB1/MDR1, ABCB11/BSEP, ABCC2/MRP2, ABCG2/BCRP. Alternatively, solute carrier (SLC) transporters may include one or more of the group consisting of: SLCO1B1/OATP1B1, SLCO1B3/OATP1 B3, SLCO1A2/OATP1A2, SLCO2B1/OATP2B1, SLC22A1/OCT1, SLC22A7/OAT2, and SLC47A1/MATE1.

It will be appreciated that in certain instances a specified xenobiotic compound, molecule or composition may act as a substrate for several drug metabolizing enzymes and/or drug clearance proteins. It is an advantage of the present invention that a virtual physiologically based pharmacokinetic (PBPK) model may be constructed that incorporates the relative contributions of a plurality of enzymes and/or proteins, such as those described herein, that are involved in the clearance and/or metabolism of the specified xenobiotic. The plurality of xenobiotic/drug clearance or metabolizing enzymes or proteins that inform the virtual PBPK model may comprise a plurality of CYPs, or a combination of one or more CYPs and one or more non-CYPs, suitably one or more CYPs and one or more transferases and/or transporters.

As used herein, the phrases “organ marker genes” or “marker genes” refer to genes expressed principally in organs associated with drug/xenobiotic clearance, suitably the liver, consistently and at relatively high levels. By “relatively high levels” it is meant that the expression profile of a given marker gene is expressed, usually constitutively, at readily detectable and quantifiable levels. In the present invention, the amount of these marker genes as measured in circulating RNA may be used as an indicator for the degree of shedding taking place in a certain individual. Thus these data on marker gene level may be used as a ‘benchmark’ to determine an average baseline of shedding in an individual, showing a basal level of organ gene expression. Such data may be used to reduce variation in correlation of other mRNA sample levels to expression levels of genes in the organ.

Suitably, organ marker genes indicative of the liver may be selected from any, some or all of: A1BG (Alpha-1-B glycoprotein), AHSG (alpha-2-HS-glycoprotein), ALB (Albumin), APOA2 (Apolipoprotein A-II), C9 (Complement component 9), CFHR2 (Complement factor H-related 5), F2 (Coagulation factor II (thrombin)), F9 (Coagulation factor IX), HPX (Hemopexin), SPP2 (Secreted phosphoprotein 2), TF (Transferrin), MBL2 (mannose-binding lectin (protein C) 2), SERPINC1 (Serpin peptidase inhibitor, clade C (antithrombin), member 1) and FGB (Fibrinogen beta chain).

Organ marker genes indicative of the gut include: FABP6 (fatty acid binding protein 6); VIL1 (villin 1); LCT (lactase); DEFA6 (defensin alpha 6); DEFA5 (defensin alpha 5); CCL25 (C-C motif chemokine ligand 25); RBP2 (retinol binding protein 2); APOA4 (apolipoprotein A4); REG3A (regenerating family member 3 alpha); FABP6 (fatty acid binding protein 6); MEP1B (meprin A subunit beta); ALPI (alkaline phosphatase, intestinal); and CPO (carboxypeptidase O); those indicative of CNS include: OPALIN (oligodendrocytic myelin paranodal and inner loop protein); GFAP (glial fibrillary acidic protein); OMG (oligodendrocyte myelin glycoprotein); OLIG1/2 (oligodendrocyte transcription factor 1/2); GRIN1 (glutamate ionotropic receptor NMDA type subunit 1); NEUROD6 (neuronal differentiation 6); CREG2 (cellular repressor of E1A stimulated genes 2); NEUROD2 (neuronal differentiation 2); ZDHHC22 (zinc finger DHHC-type containing 22; KCNJ9 (potassium voltage-gated channel subfamily J member 9); GPM6A (glycoprotein M6A); PLP1 (proteolipid protein 1); and MBP (myelin basic protein)]; and those specific to kidney include: UMOD (uromodulin); KCNJ1 (potassium voltage-gated channel subfamily J member 1); TMEM174 (transmembrane protein 174); NPHS2 (podocin); AQP2 (aquaporin 2); TMEM52B (transmembrane protein 52B); CTXN3 (Cortexin 3); TMEM27 (transmembrane protein 27); SOST (sclerostin); and CALB1 (Calbindin 1).

It will be appreciated that the aforementioned lists are not exhaustive and a plurality of alternative organ or tissue specific marker genes may be selected from, for example, the organ-specific or tissue-specific proteomes respectively. Often organ specific marker genes will comprise constitutively expressed genes that show relatively constant or consistent levels of expression with low or predictable variance over time in tissues having normal pathology—e.g. housekeeping genes. Organ or tissue phenotype marker genes may be comprised within a ‘panel’ that comprises a plurality of such genes. Typically, a panel of organ/tissue marker genes would include not less than six, suitably not less than eight, typically around ten and optionally not less than twelve genes expressed principally in the specified organ or tissue, consistently and at relatively high levels. Such marker genes may be derived from healthy tissues or organs, or they may be derived from diseased tissues/organs. In an embodiment of the invention, the tissue comprises neoplastic tissue, which may be benign or malignant.

As used herein the term “shedding” is used to describe the process of mRNA release by cells from organs or tissues, such as liver hepatocytes, into a bodily fluid, in microvesicles, exosomes, or otherwise as cell free mRNA. mRNA shedding can vary in magnitude between subjects or within the same subject depending on, for example, disease state, and affects the correlation between the levels of a particular RNA detected in the blood, plasma or other sample, and the levels of the same mRNA in the cells and tissue of the organ, such as the liver. The term “RNA shedding” is used as a synonym.

A “shedding coefficient”, organ “shedding correction factor” or “SCF” refers to a scaling factor for an individual which relates to the amount of shedding by their hepatocytes. A “fast shedder” will shed more RNA for the same amount of gene expression than will a “slow shedder”, and thus the SCF for such individuals will differ. It is contemplated that the SCF can be calculated from the quantified levels of the cell free RNA (cfRNA) of one or a plurality of organ/tissue marker genes, for example from 1, 2, 3, 4, 5, 6, 7, 8, 9, 10, 11, 12, 14 or more genes. The phenomenon of shedding correction is identified and exemplified in co-pending International Patent application No. PCT/US2019/24379 (the contents of which are incorporated herein by reference).

If a subset of N organ or tissue specific markers are used the SCF can be calculated as follows.

$$SCF = {\sum }_{i = 1}^{N} {[ cFRNA ]}_{{Marker}_{i}} / ( N \times {[ cfRNA ]}_{TOTAL} )$$

Identification of the SCF for a given individual subject from which a liquid biopsy has been obtained is, therefore, a key step in embodiments of the present invention. The SCF allows for the further analysis of the exosomal mRNA in the biopsy in order to identify the abundance of a protein encoded by the mRNA in the organ/tissue of origin for the subject. Without the ability to correct for RNA shedding the derivation of transcriptomic information from any liquid biopsy is made substantially more difficult and potentially even meaningless.

Pharmacokinetics (PK) is the study of what happens to a drug when it is administered to and passes through the various organ and tissue compartments within the body of a subject. Drug absorption, distribution, and elimination are subject to multiple interactions dependent in part upon the biological action of each organ on a drug, partitioning of the drug to these organs and tissue volumes (compartments) and blood flows. The absorption (rate and extent of bioavailability), distribution/localisation, metabolism and excretion (ADME), biotransformation and toxicity profiles of any given pharmaceutical or other xenobiotic compound are key deterministic measures of subsequent pharmacodynamics (action of the drug on body) necessary to achieve efficacy without major safety issues prior to an authorisation for use in medicine.

However, the majority of studies and associated models aggregate across populations leading to predictive models and virtual simulations based upon an average response or response in the average representative of a stratified group. This approach is sometimes referred to as population based pharmacokinetic (popPK) modelling. PopPK methodology relies on mathematical models to describe PK data. Clinicians and drug developers often utilise popPK models in order to help guide decision-making across all phases of drug development. As such, the pharmaceutical industry has become increasingly reliant upon popPK models to generate efficacy and safety data in support of applications for regulatory marketing authorisations (i.e., new drug applications ‘NDAs’ and biologics license applications ‘BLAs’).

In an embodiment, the present invention is based in part upon an assay that determines the level of one or more mRNAs coding for xenobiotic metabolizing enzymes or clearance proteins originating from an organ/tissue in a liquid biopsy, such as a biological blood sample, via a quantitative analysis of the sample. This analysis establishes a correlation from these determined levels to the levels—or abundance/concentration—of xenobiotic metabolizing and/or transporting enzymes in the organ, and membrane transporters and transferase enzymes in other tissues of an individual. The abundance relationship may be supplemented by reference to standardized curves or log tables generated by comparison of matched samples comprising a liquid biopsy and a tissue biopsy from one or more reference individuals. Generally, the matched samples are obtained from the same individual. In this way, the concentration of mRNA for a clearance or metabolizing protein present in the liquid biopsy is capable of direct relation to the corresponding abundance/concentration of the protein in the organ/tissue of origin. This in turn allows for the xenobiotic clearance capacity of the organ/tissue of origin to be estimated with a high degree of accuracy. Information regarding the xenobiotic clearance capacity of an organ or tissue for a given protein having pharmacokinetic activity (such as a CYP, transferase or transporter) represents one of the building blocks of bottom up PBPK models.

In embodiments of the invention, the xenobiotic metabolizing enzymes or clearance proteins may be predominantly, or even exclusively, expressed within a single tissue or organ or origin. This enables a high level of confidence that the adjusted level of circulating mRNA determined according to the methods described correlate well to the abundance of the encoded protein in the tissue of origin. An example of a protein having relatively restricted expression is CYP1A2, which is predominantly expressed in the liver. In instances where a protein is expressed by more than one tissue or organ of origin the calculated concentration level of that protein can be weighted accordingly in relation to the known levels of protein abundance in the tissues of origin. For example, CYP3A may be expressed in both the gut and the liver although the net intestinal abundance of CYP3A in the gut is only around 1% of that in the liver (Yang et al. Clinical pharmacology & therapeutics (2004);76(4); and Yang et al. Current Drug Metabolism (2007) 8, 676-684).

Variability of drug response between individuals is an important consideration in clinical medicine. One major determinant of drug response variability is hepatic CYP-mediated drug metabolism due to polymorphism and allelic variation, and difference in expression levels across populations. Sex differences are also known to affect drug metabolism with females more than 50 to 75% more likely to observe adverse effects. Other variations may be due to polymorphism or difference in expression levels of other key proteins involved in drug clearance including membrane transporter proteins and transferase enzymes. Hence, the analytical data generated by the inventive methods and apparatus provides a key advantage that enables the construction of significantly improved personalised as well as population based pharmacokinetic computer models. These models and simulations may be used in the design of improved clinical trials, incorporated into the decision of better dosage regimens, or used to predict and inform personalised medicine choices.

The individuals tested or treated according to various embodiments of the invention may be healthy or diseased, and human or animal patients. In veterinary contexts, the drug clearance models may require suitable adaptation, although the underlying principles of the invention are consistent. The term “animal” may include mammals such as cats; dogs; mice; guinea pigs; rabbits; primates; horses; as well as livestock including cattle; pigs; sheep; and goats.

For individuals or populations suffering from disease or abnormal pathology the current development of PBPK models is difficult due to a lack of available data to better characterize the altered physiological state. The lack of in vivo data for generating models for individuals and populations suffering from cancer is particularly evident. Prediction of drug clearance during infection and inflammation is a further important consideration for disease state modelling, given that this altered state leads to downregulation of metabolizing enzymes such as CYPs in the liver and gut due to elevated levels of pro-inflammatory cytokines. Since the design of personalised dosage regimens for drugs in individual patients is assigned based on the extent of drug clearance for that individual to avoid overexposure, the effect of disease such as renal or hepatic impairment, which can significantly reduce clearance, is crucial. Hence, in embodiments of the invention a process is provided for establishing a virtual model of metabolic xenobiotic clearance in an individual subject or a population of individual subjects, wherein the subject(s) are suffering from disease or altered physiological state associated with an abnormal pathology.

According to embodiments of the present invention personalised virtual models of metabolic xenobiotic clearance may be utilised in the development of more personalised dosage regimens. Such personalised dosage regimens may be used in methods of treatment of individual subjects in need thereof. In specific embodiments, dosage regimens may be formulated for the treatment of a range of diseases including, but not limited to, cancer; inflammatory disease; auto-immune disorders; allergy; metabolic diseases, including metabolic deficiency; degenerative diseases, including neurodegenerative diseases (e.g. Alzheimer's, Parkinson's, ALS, multiple sclerosis, Huntington's); psychiatric disorders; infection, including chronic or acute infection from bacterial, viral, fungal or parasitic pathogens.

In an embodiment of the invention, the levels of mRNAs—suitably cell free mRNAs—that encode drug metabolizing and transporting proteins, including CYPs, transport proteins and transferases, are measured in a liquid biopsy, suitably a blood sample. The concentration or amount of each mRNA in the blood sample thereby correlates to an amount/concentration/abundance of a drug clearance protein, for example, an enzyme or transporter, in the organ or tissue of the individual from which the mRNA originated. The prediction of amount/concentration/abundance of a drug clearance protein based upon the amount or concentration of the mRNA present in the liquid biopsy can be made by consultation with a calibration curve or log table, for instance.

The transcriptomics profile can be used to build a virtual system to provide an in silico model for an individual subject or if combined with a plurality other individuals to provide virtual population, or sub-population. Such models can be tested to predict the individual or a population's capacity for clearance with one or more xenobiotic compounds. The system can be further refined by the addition of information derived from biomarkers found within the same or a different sample, and/or with other physiological and/or epidemiological information, which may be gathered by questionnaire, interview, health professional analysis, measurement with medical diagnostic equipment, or similar. The virtual individual or population can also be tested for undesirable interactions that might occur between combinations of xenobiotics. For example, in the field of pharmacology such interactions are referred to as drug-drug interactions (DDIs). Conventionally model based DDIs are studied by either a Mechanistic Dynamic interaction Model (MDM) based on in vitro data plugged into an appropriate PBPK model or a Mechanistic Static interaction Model based on in vivo data (IMSM). A problem with IMSM models is that they can be constrained by availability of reliable in vivo data. For instance, it is known that genetic polymorphism can have a significant effect upon cytochrome metabolism and, thus, upon any DDIs that may occur in an individual (Tod et al. AAPS J., 2013 October; 15(4): 1242-1252). Most IMSM models require multiple interactions of compounds to be undertaken in many individuals. By enabling the generation of additional in vivo data from a liquid biopsy, it is an advantage of the present invention that it allows for the development of better IMSMs as well as a hybrid approach in which an MDM can be further informed by real world in vivo data that feeds into and allows for the generation of highly bespoke PBPK models.

Isolation of exosomal or microvesicular components from a liquid biopsy may be performed using techniques such as spin column chromatography, immunoaffinity, membrane affinity, affinity labelled microbeads, precipitation and/or ultracentrifugation with a density gradient. Optimisation or choice of techniques will depend upon factors such as sample volume versus the type of liquid biopsy being handled. In an example of the invention described in more detail below, RNA comprised within exosomal or microvesicular components of a blood plasma liquid biopsy are isolated using a membrane affinity column utilising selective binding to a silica-based membrane.

Biomarker levels within a liquid biopsy sample may be determined by a range of techniques including macromolecule microarray analysis, mass spectrometry (MS) proteomic profiling, quantitative RT-PCR, ELISA or other antibody-based assays, and chromatographic or spectrophotometric techniques.

RNA transcripts that are isolated from the liquid biopsy sample may be detected by a range of methods, including but not limited to polymerase chain reaction (PCR), reverse transcription polymerase chain reaction (RT-PCR), quantitative real time polymerase chain reaction (Q-PCR), gel electrophoresis, capillary electrophoresis, mass spectrometry, fluorescence detection, ultraviolet spectrometry, DNA hybridization, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof. RNA may be reverse-transcribed by any suitable means to produce cDNA before analysis in any combination with the above. RNA levels can be determined by use of nucleic acid hybridisation arrays, next generation sequencing approaches or real-time PCR.

Bioanalysis of RNA samples may also occur using RNA sequencing such as by use of a single-end sequencing-by-synthesis reaction e.g. Ampliseq (Thermo Fisher, USA), HiSeq 2500 or NextSeq 550Dx Systems (Illumina, USA).

DNA arrays are solid supports upon which a collection of gene-specific nucleic acids have been placed at defined locations. In array analysis, a nucleic acid-containing sample is labelled and then allowed to hybridise with the gene-specific targets on the array. Based on the amount of nucleic acid from the sample hybridised to target on the array, information is gained about the specific nucleic acid composition of the sample. Array analysis, according to the present invention, involves isolating total RNA from a sample comprising cells or microvesicular material, converting the RNA samples to labelled cDNA via a reverse transcription step, hybridising the labelled cDNA to identical arrays (such as via either a nylon membrane or glass slide solid support), removing any unhybridized cDNA, detecting and quantitating the hybridised cDNA, and determining the quantitative data (e.g. the levels of biomarkers present) from the various samples.

Real-time or quantitative PCR refers to a method which monitors the replication of a nucleotide sample in real-time during the PCR reaction. As well as the normal components, the reaction mixture contains fluorescent probes which may hybridise to any double-stranded nucleotide sequence or else to a specifically chosen complementary sequence. The signal from the fluorescent probes therefore correlates with the number of the target sequences which have been produced during the reaction and can be used to determine the quantity of the target sequence in the original sample.

Analysis of RNA levels (e.g. quantifying the RNA concentration or amount) within the liquid biopsy sample allows for adjustment of the generic settings (standard baseline settings) for the simulation algorithm to correspond to those of the individual subject. In this regard the methods of the invention allow for the determination of the biomarker profile generated for the individual to be correlated with the corresponding levels of drug metabolizing, transporting and clearance proteins within the tissues of said individual, such as in the liver, kidney, CNS or gut and on the surface of the target cell. This step comprises quantifying the levels (including concentration/amount/activity) of at least one biomarker within the sample from the individual which is correlated to at least one level of a drug metabolizing, transporting and clearance protein or enzyme within the individual's tissues via a defined correlation function or algorithm. Hence, the model according to the invention is able to determine a correlation between first input data in the form of a biomarker profile from the sample and the activity/concentration/amount level of drug metabolizing, transporting and clearance protein or enzyme within the individual's tissues. This enables the production of an augmented profile that comprises baseline biomarker data from the original sample together with the correlated (or predicted) level of drug metabolizing, transporting and clearance protein or enzyme within the individual's tissues. The augmented first input data is used to define the starting points for the simulation model of the invention, in terms of baseline levels of drug metabolizing, transporting and clearance protein or enzyme within the individual's tissues, optionally in combination with gene identity and expression data including allelic variation and whether certain genes are either up- or down-regulated compared to average (i.e. mean or median) levels within a given population. The term “down-regulated” as used herein denotes a process resulting in decreased expression of one or more genes and/or the proteins encoded by those genes. “Up-regulated” denotes an increase in gene expression and corresponding protein expression.

Additional factors may have a bearing on drug response. These characteristics may be determined by the measurement of biomarkers in a sample, which can be the same or different sample as the liquid biopsy sample used for determination of the one or more mRNAs coding for drug metabolizing enzymes or drug clearance proteins. For example, allelic variations of CYPs, transporter genes or transferases, or any other relevant gene, may be determined from genomic DNA isolated from a liquid biopsy sample or any of a number of biological samples. This can include information not able to be derived from mRNA sequences, such as intron data, epigenetic information and the presence and activity of genomic regulatory features such as promoters, repressors, and so on.

Non-gene expression parameters which may also be relevant for determining drug response may include parameters which can be determined by measurement of biomarkers in one or more liquid biopsy sample, and/or can include physiological and epidemiological information collected by other means. In some embodiments of any aspects of the invention, one or more non-gene expression parameters may be selected from the group consisting of: ethnicity; genotype; age; age group classification; gender; smoking status; presence of chronic disease, including renal impairment, diabetes (type I or type II) or liver cirrhosis; body mass index (BMI); body adiposity index (BAI) or other equivalent measurements of body fat content; waist circumference measurement; waist-to-hip ratio; hydrostatic weighting; average alcohol consumption; pregnancy; allergy status; blood pressure; total blood lipids (e.g. cholesterol); average resting heartbeat; ECG interval measurements including QT interval, QRS duration, and PR intervals; general medical history; familial medical history; or combinations thereof. Such additional parameters may be used to further refine any model, algorithm, simulation or prediction produced by the invention, improving accuracy.

Embodiments of the present invention provide a method that is used to build a robust computer (in silico) predictive model of drug metabolism, in particular drug distribution and clearance, for a specified individual subject. In this way a computer-based model of drug clearance can be matched to any given individual, following a simple blood test, and thereby provides an accurate personal prediction of an individual's capacity to metabolize and/or clear a given drug, xenobiotic, or combination of drugs or xenobiotics. The so-called Virtual Twin model is incorporated into a computer implemented system that can be utilised by, for example, clinicians, academics, patients and pharmaceutical researchers.

According to an embodiment of the invention the method comprises the steps of obtaining a liquid biopsy sample from an individual. The liquid biopsy may suitably comprise a bodily fluid such as any one or more of: blood, urine, saliva, semen, tears, lymphatic fluid, cerebrospinal fluid, bile, stool or a mucus secretion. This sample can be obtained via a minimally invasive route, and can include deriving blood components such as plasma, serum or other sample from a whole blood liquid biopsy sample. The sample is analysed quantitatively to determine the levels of one or more, typically a plurality, of mRNAs coding for drug metabolizing enzymes, transporter proteins or other clearance proteins in order to derive a profile of the said individual's circulating mRNA. The sample is also analysed to determine the levels of one or more organ specific marker genes in the sample in order to give a subject-specific organ shedding correction factor (SCF). The SCF is used to provide a baseline for the rate or amount of circulating mRNA shed by the organ and is thus used as an adjustment factor in order to correct the profile of the individual's circulating mRNA.

The corrected profile defines first input data, which first input data is then used to inform a computer-based model of drug pharmacokinetics. The calibration step thereby enables the creation of an individual model of drug absorption; distribution; localization; biotransformation; and/or excretion. This individual model accurately predicts the pharmacokinetics and pharmacodynamics of drug metabolism and clearance, for a specified xenobiotic or pharmaceutical compound or combination of the same. Hence, according to one embodiment, the invention provides a robust model that simulates the individual pharmacokinetics for a specified subject based upon a ‘bottom up’ approach. This contrasts with established ‘top-down’ population-based (popPK) models which require pre-existing models for any given drug against which the individual subject is then compared.

In some embodiments the method further comprises quantitatively analysing a sample to determine the levels of one or more, typically a plurality, of biomarkers present within the sample in order to derive a profile of the said individual's biomarker(s). The sample may be the same or different to that sample for determining circulating RNA, and as such may further include the steps of obtaining a second biological sample from an individual. The sample may be obtained in any suitable way, but may again be obtained via a minimally invasive route, such as a blood, cheek swab, saliva, stool or urine sample. The profile defines biomarker input data, which biomarker input data is then used to further calibrate the computer-based model of drug clearance.

In some embodiments, physiological and/or epidemiological information to obtain non-gene expression data not derivable from sample biomarkers may be obtained from an individual, in order to derive a physiological and/or epidemiological profile of the said individual. Such information may include ethnicity; age; gender; smoking status; body mass index (BMI); body adiposity index (BAI) or other equivalent measurements of body fat content; waist circumference measurement; waist-to-hip ratio; allergy status; blood pressure; average resting heartbeat; ECG interval measurements including QT interval, QRS duration, and PR intervals; general medical history; familial medical history; or combinations thereof. The profile defines personal input data, which is then used to further calibrate the computer-based model of drug clearance.

One embodiment of the present invention provides a sophisticated platform for the analysis of pharmacokinetic outcomes, drug-drug interactions (so-called DDIs) and tissue-specific responses in a given individual, resulting in a comprehensive physiologically-based pharmacokinetic (PBPK) model. PBPK models can comprise nested compartments that represent different tissue functionalities and cell types within an organ system. When assembled, the levels of hierarchical complexity allow for modelling of molecularly-driven events, such as specific metabolic pathways. The blood flows and partition coefficients that link the compartments—i.e. the organ systems—together mathematically are estimated from animal, in vitro data, and clinical data. The parameters and compartments are then optimized to fit the model to existing data.

Hence, the present invention provides a significant advantage over and enhancement of prior art modelling systems that are largely based upon population level, animal or entirely in vitro based responses. In contrast, according to specific embodiments the present invention provides a virtual mimic, also referred to as a “Virtual Twin”, for an individual. This Virtual Twin may represent an in silico model that is configured so as to represent an entirely personalised PBPK model for a given individual. The model may represent the consolidation of multiple data inputs from a variety of sources, including the physiological and/or epidemiological information described above, the genotype as well as a SCF. This approach facilitates the growth of personalised medicine solutions, improved design of dosage regimens and the identification of potentially harmful side effects before a drug, xenobiotic, or combination of same is administered. In addition the present invention may provide a direct correlation between the levels of circulating mRNA present in, for example, the blood with the level (e.g. abundance) of drug metabolizing, transporting and clearance proteins within tissues, such as the liver, in that individual. Previous approaches have only looked to correlate mRNA and/or biomarker levels with estimates of enzyme activity against a specified probe compound, and as a result have struggled to find utility outside of the very limited probe-enzyme system described.

The virtual simulator may also incorporate an in vitro to in vivo extrapolation (IVIVE) approach to further inform the model. The IVIVE approach establishes virtual populations by building up mechanistic and physiologically based pharmacokinetic (PBPK) models. These models incorporate identified variabilities in demographic and biological (genetic and environmental) components linked to drug-specific physicochemical properties (for example, aqueous and lipid solubilities) and in vitro data on absorption, metabolism and transport. The covariate relationships embedded in such models can be complex and nonlinear and can be difficult to resolve by simple linear covariate analysis. The primary advantage of the IVIVE approach is that it maximizes the value of all in vitro information previously generated during drug discovery and preclinical development.

The algorithm of an embodiment of the invention may include consideration of SCF-corrected input data comprising data pairs or even data clusters. Suitably, data derived from mRNA analysis, such as gene expression data for drug clearance genes, may be categorised further via one or more additional gene and non-gene expression parameters, which may be derived from analysis of biomarkers detected in one or more biological samples. Non-gene expression parameters may include physiological and epidemiological information. In some embodiments of any aspects of the invention, one or more non-gene expression parameters may be selected from the group consisting of: ethnicity; genotype; age; age group classification; gender; smoking status; presence of chronic disease, including renal impairment, diabetes or liver cirrhosis; body mass index (BMI); body adiposity index (BAI) or other equivalent measurements of body fat content; waist circumference measurement; waist-to-hip ratio; hydrostatic weighting; average alcohol consumption; pregnancy; allergy status; blood pressure; total blood lipids (e.g. cholesterol); average resting heartbeat; ECG interval measurements including QT interval, QRS duration, and PR intervals; or combinations thereof.

In a specific embodiment of the invention, the described methods can be implemented via one or more computer systems. According to a further embodiment, an apparatus comprising one or more memories and one or more processors is provided, wherein the one or more memories and the one or more processors are in electronic communication with each other, the one or more memories tangibly encoding a set of instructions for implementing the described methods of the invention. In another embodiment the invention provides a computer readable medium containing program instruction for implementing the method of the invention, wherein execution of the program instructions by a controller comprising one or more processors of a computer system causes the one or more processors to carry out the steps as described herein. Suitably, the data may be stored in a database, and accessed via a server. Suitably, the server is provided with communication modules to receive and send information, and processing modules to carry out the steps described herein. In some embodiments, the data is provided through a cloud service. In particular embodiments, the method is accessible as a web service. In some embodiments, users may access the service for recordal or retrieval of scores via a website, in a browser. Networking of computers permits various aspects of the invention to be carried out, stored in, and shared amongst one or more computer systems locally and at remote sites. Hence, two or more computer systems may be linked using wired or wireless means and may communicate with one another or with other computer systems directly and/or using a publicly-available networking system such as the Internet.

Suitably, the computer system includes at least: an input device, an output device, a storage medium, and a microprocessor). Possible input devices include a keyboard, a computer mouse, a touch screen, and the like. Output devices computer monitor, a liquid-crystal display (LCD), light emitting diode (LED or OLED) computer monitor, virtual reality (VR) headset and the like. In addition, information can be output to a user, a user interface device (e.g. tablet PC, mobile phone), a computer-readable storage medium, or another local or networked computer. Storage media include various types of memory such as a hard disk, RAM, flash memory, and other magnetic, optical, physical, or electronic memory devices. The microprocessor is a computer microprocessor (e.g. CPU) for performing calculations and directing other functions for performing input, output, calculation, and display of data. In one embodiment of the invention, the computer processor may comprise an artificial neural network (ANN). In a further embodiment of the invention the computer processor may comprise a machine learning algorithm, suitably a machine learning algorithm that has been trained against one or more appropriate data sets.

The modelling platform of the invention allows for accurate in silico simulation of pharmacodynamic and pharmacokinetic responses by combining two primary classes of data. The first class of data is the corrected first input data in the form of circulating mRNA expression for xenobiotic clearance proteins, corrected (such as SCF-corrected) and augmented information (as described above) related to the individual. The second class of data is termed “second input data” and relates to the identity of the drug, compound or substance under test. If drug-drug interactions (DDI) are under consideration then there may be a plurality of second input data. These two types of data may be conveniently stored within XML-based or JavaScript-based file format that can be viewed and accessed via the system graphical user interface (GUI) as well as other tools such as Microsoft Edge™ (Microsoft Corp., Redmond (Wash.), USA) or Google Chrome (Google LLC, Mountain View (Calif.), USA). The schema of these files is designed to allow forward compatibility of files over time such that future release versions and new parameters may be added without disrupting what already exists. This allows files created with a current version of the simulator and to be used with later versions when they are released where any possible missing values are automatically replaced with default values. Files may contain a degree of meta data showing varying information including the software version used to create the file.

The corrected first input data and second input data provides the baseline information for initiating a simulation of drug clearance for a given individual. However, it may also be necessary or desirable to create a workspace that provides contextual information about conditions in which the trial is to be undertaken. The workspace file may also be XML or JavaScript-based; however, this time it acts as a container for first and second input data as well as any trial/simulation information and user defined settings. The workspace may also be used as a snapshot of the running condition of any simulation. In other words, to reproduce any simulation exactly, all that is needed is a copy of the workspace taken at the time the simulation was run.

The simulation algorithm of the invention handles a multiplicity of pharmacokinetic (PK) model combinations, including:
(1) Administration of single (small and large molecules) or multiple chemical moieties,
(2) Different absorption models, namely one-compartment, enhanced Compartmental Absorption and Transit (CAT), and Advanced Dissolution, Absorption and Metabolism (ADAM) models,
(3) Different distribution models such as minimal and full PBPK models with different perfusion- and permeability-limited models, including multi-compartment organ, kidney, blood-brain-barrier, intestinal degradation models and an additional multi-compartment user defined organ/tissue,
(4) Modelling of a plurality of metabolites, and
(5) Pathology or disease state of the subject or population being modelled, e.g. whether the subject or population are healthy or not.

According to one embodiment of the invention, PBPK model algorithms are built using ordinary differential equations (ODE) (for example see Jamei Met al., Expert Opin Drug Metab Toxicol. 2009 Feb; 5(2):211-23; and Jamei et al. AAPS J. 2009 Jun; 11(2): 225-237; Nagar et al. Mol Pharm. 2017 Sep 5; 14(9): 3069-3086).

The methods of the invention are particularly useful in contributing to improved construction of PBPK models by providing better understanding over how abundance of drug clearance proteins can vary between individuals. This is important as a result of the increased dependency on PBPK models to address regulatory questions as well as their ability to minimize ethical and technical difficulties associated with pharmacokinetic and toxicology experiments for special patient populations. Hence, the invention provides, in one embodiment, an improved method for creation of computer based models for the determination of clearance of a given xenobiotic molecule (e.g. drug or biological therapeutic) from individual, or when cumulative data is provided, from a population of individuals. The ease of liquid biopsy, which is far less invasive then solid tissue biopsy sampling, is a major factor in contributing to improved construction of computer models that show utility in drug development and clinical trial design. It also enables new models to be created for use in distinct cohorts such as for neonatal and paediatrics as well as in smaller ethnically distinct populations, or for rare diseases, by way of non-limiting example.

The exposure of an individual to a certain drug can be measured by the area under the concentration time curve (AUC). The AUC after administration through any non-parenteral route (such as an oral dose) is dependent on the proportion of the dose that is absorbed and is subsequently available in the systemic circulation. In the case of oral drug administration (the most common route for drug intake), this involves release of the drug from the formulation, passage through the gut wall and then through the liver. The bioavailability of the drug (F) together with the clearance (CL) and the dose of the drug (D) will determine the overall exposure (AUC) according to the following equation 1 below:

$$AUC = \frac{F \times Dose}{CL}$$

Total clearance (CL) is defined as the volume of blood completely cleared of drug per unit time and encompasses clearance by the liver, the kidneys and biliary excretion (in the absence of re-absorption from the gut). Although exposure to the drug is determined only by the dose, clearance and bioavailability, varying shapes of concentration-time profile can occur for a given exposure when the rate of entry (absorption rate, infusion rate etc.) and rate of elimination are changed. Elimination rate is a function of clearance and distribution characteristics.

Since the majority of drugs currently on the market are lipophilic, metabolism is a major route of elimination from the body. It should be noted that overall metabolic clearance is not usually a simple linear function of the organ but it is also dependent on the delivery of the free drug to the site of metabolism. By way of example: hepatic clearance, distribution and metabolism may be determined by factors such as hepatic blood flow, plasma protein and red blood cell binding, and the effects of influx into or efflux from hepatocytes. In vivo intrinsic organ clearance has been extrapolated from in vitro models using human liver microsomes/exosomes or human hepatocytes in culture. However, to determine whole organ or even systemic clearance requires the combination of intrinsic clearance rates for multiple drug clearance/metabolising enzymes and transporters in different organs and tissues. For each individual the levels of these enzymes will vary, thus resulting in a different level of clearance for that individual.

An expression that estimates the net intrinsic metabolic clearance in total by the whole liver (CLu_{H,int}) from data obtained with recombinantly expressed CYP enzymes is given by equation 2, below:

$$[ {\sum }_{j = 1}^{n} ( {\sum }_{i = 1}^{n} {ISEF}_{j i} \times \frac{{V}_{ma xi} ( rh {CYP}_{i} ) \times {CYP}_{j} abundance}{{K}_{m i} ( {rhCYP}_{j} )} ) ] \times MPPGL \times Liver weight$$

where there are i metabolic pathways for each of j CYPs, rh indicates recombinantly expressed enzyme, Vmax is the maximum rate of metabolism by an individual CYP, Km is the Michaelis constant, MPPGL is the amount of microsomal protein per gram of liver and ISEF is a scaling factor that compensates for any difference in the activity per unit of enzyme between recombinant systems and hepatic enzymes. This expression indicates that inter-individual variability in hepatic intrinsic clearance can be introduced by incorporating variability in several important parameters. One key parameter influencing this model is the liver abundance (e.g. the level) of each CYP in the individual. Other parameters such as MPPGL and liver weight can be estimated based upon height, weight and age of the individual. However, it is the differences in in metabolism that result from CYP abundance, as well as functional, genetic polymorphisms that can be accommodated by knowing the frequency of different genotypes, and by modifying either the enzyme abundance or the intrinsic enzyme activity. Data on changes in the abundance and/or activity of different xenobiotic clearance proteins, such as CYPs, is incorporated into the virtual model of the present invention in order to predict hepatic clearance in individuals.

PBPK models for tissues other than liver may include components that describe ingestion and/or inhalation, homeostatic control (e.g. gastrointestinal absorption), and delivery to target sites within tissues and organs of the body.

Hence, the virtual in silico model of an embodiment of the invention comprises algorithms that are able to incorporate in vitro data on drug metabolism/clearance and inter-individual variability that is relevant to drug metabolism/clearance in the tissues of the individual concerned. Optionally, the virtual model of the invention may further incorporate allometric scaling models. Allometric scaling methodology attempts to predict mean clearance values in humans from those observed in animal species by scaling for body size. The use of an approach that incorporates IVIVE in addition to allometric scaling has the added advantage of being able to assess the likely individual allelic variability in clearance. For example, some allelic variations of CYP enzymes show decreased catalytic activity compared to wild type and, thus, having knowledge of an individual's specific genotype enables in vitro data on kinetics to be used to estimate in vivo clearance.

Hence, by incorporating in vitro information on enzyme inhibition constants (competitive or non-competitive inhibition) into the virtual model of embodiments of the present invention it is possible to predict the extent of metabolic drug-drug interactions (mDDI) in vivo for any given individual.

Creating a simulator that provides accurate clearance and/or metabolic capacity values for an individual requires the consideration of multiple parameters—amongst other things: size of organ, genotype of certain enzymes, kidney function etc. However, of critical importance is the affinity of a given xenobiotic compound (e.g. drug) for the drug clearance proteins present and efficiency of each molecule of protein in handling each molecule of xenobiotic compound. This relationship is described as the K_{cat} and is based on intrinsic clearance of the drug by a given enzyme. By way of example, as described previously, certain drugs are metabolized by particular CYPs, transferases and operate through specific membrane transporters, as well as specific combinations of these drug clearance proteins. The K_{cat} for each CYP or transferase with a given drug is a key determining factor ascertained during clinical trials of any pharmaceutical compound. Hence, if the K_{cat} and/or CLᵢₙₜ is a key parameter that is determined in all modern drug development programs, then by estimating the abundance of the relevant drug clearance proteins in an individual's organ, such as the liver, it is possible to predict the clearance of that drug in that individual. The virtual simulator, of one embodiment of the invention, operates by summing up the various clearances and putting them through the appropriate models of the organ, that consider inter alia the limitations of blood flow and availability of free drug concentration in the blood to the organ. Previous attempts at creating such models have typically resulted in low success because they relied upon unmatched samples or estimates of activity based upon literature referenced population averaged levels of abundance. The compromises introduced when using parameters that are averaged across populations leads to an unacceptably high coefficient of variation of AUC (% CV) in any given dosage regimen. Clearly this increases the difficulty in achieving true precision dosing.

Accordingly, in a specific embodiment of the invention a dosage regimen is provided, in which parameters related to the administration of a drug comprising a pharmaceutical compound or a biological therapeutic agent to a subject are determined in conjunction with that individual's clearance capacity for the compound or agent. More specifically, a liquid biopsy may be obtained from a subject and cfRNA_{TOTAL} analysis performed. From the cfRNA_{TOTAL} analysis a SCF for the subject is able to be determined. PBPK and popPK clearance models are understood for a wide range of approved pharmaceutical compounds and compound classes. In particular, the specific drug metabolising CYPs, transferases and transporters etc. that are relevant for clearance of most compounds and agents and, therefore, the one or more clearance proteins that constitute [cfRNA]_{Target} for the given drug may be identified. The normalized [cfRNA]_{Target} for the specific clearance protein(s) may be determined from the cfRNA_{TOTAL} thereby enabling the abundance of the specific clearance protein(s) within the relevant organs of a given individual to be ascertained. It will be appreciated that having information of this type for any given individual in relation to a proposed a pharmaceutical compound or a biological therapeutic agent that is to be administered enables a precision dosing regimen to be formulated for that individual for the specific drug.

In a further embodiment of the invention, the individual subject may be a recipient of two or more pharmaceutical compounds or biological therapeutic agents, possibly as a result of a combination therapy or otherwise, in this instance there exists a risk of an adverse DDI. Hence, the individual subject's clearance and/or metabolic capacity for each administered therapeutic agent may be determined as described above and then combined with existing simulation models so as to provide a prediction both of clearance capacity and DDI risk. The risk prediction and clearance capacity are used to inform the determination of a dosage regimen for the individual. It is evident that the ability to elucidate clearance capacity and incorporate this into sophisticated PBPK, PKPD and DDI models enables precision dosing at patient level that will reduce unwanted side effects, over- or under-dosing, improve bioavailability of drug and more optimised drug delivery. The consequent impact on health economics is profound as a result of optimisation of drug consumption and reduction of adverse effects across populations.

Reporting of the output data from the modelling system of the invention may be achieved via the GUI or via an output file that may comprise a .csv file or spreadsheet, such as Microsoft Excel™ (Microsoft Corp., Redmond (Wash.), USA) or Google Sheets (Google LLC., Mountain View (Calif.), USA). By way of non-limiting example, when the reporting process is implemented through the Excel Automation interface which is based on the Office Object Model. The simulation platform uses this technology to create or connect to an Excel application Component Object Model (COM) object, to manipulate and add worksheets as required. Each worksheet is a bespoke output based on the simulation input selections: each cell is effectively created individually with the selection of font (including size and weight), colour (both foreground and background), alignment of text within the cell, number format (based on the users' machine selection) as well as many other specifications.

After the output data has been rendered, graphical representations, such as dashboards, charts, pictograms or graphs may are added if applicable. These may include concentration-time profiles or, for example, pie charts of enzyme contribution which are created based on the output data comprised within the worksheet and formatted individually based on user selections such as number format, dashboard arrangement and also the colour ‘skin’ chosen before displaying the data.

In an alternative embodiment of the invention output data is comprised within a relational database. An advantage of this embodiment is that the simulator algorithm may be comprised as part of an organisational workflow as it can then write directly into a corporate database, for example. This enables formatting and visualisation and data analytics to be customised by the user.

Embodiments of the invention may also relate to an apparatus or device for performing a set of operations as defined herein, such as a set of operations that may suitably implement at least one embodiment of the present invention. The apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of medium suitable for storing electronic instructions, which may be coupled to a computer system bus. Furthermore, any computing systems referred to in the specification may include a single processor or may be architectures employing multiple processor designs for increased computing capability.

Any of the steps, operations, methods or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, methods or processes described.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

The invention is illustrated by the following non-limiting examples.

### EXAMPLES

### Example 1

The following example provides a protocol for total RNA extraction from samples of blood that can be used to determine the levels of RNA for drug metabolizing enzymes, transporters and/or marker genes in the samples, and/or RNA for the determination of biomarkers. Methods for the isolation of total protein and quantification of enzymes and transporters are described herein for the assessment of correlation between plasma RNA and tissue protein levels.

### RNA Analysis of Liquid Biopsy Comprising Blood

### A. Blood Samples

A liquid biopsy consisting of fresh peripheral venous blood may be collected from a subject and plasma isolated before further processing as described below. If required, peripheral blood mononuclear cells (PBMCs), including B and T lymphocytes, may be isolated using Ficoll-Paque PLUS (GE Healthcare Life Sciences).

Isolated plasma is stored frozen −80° C. until used for cell free RNA (cfRNA) isolation and measurement. Isolation of circulating or exosomal RNA can be done using a suitable RNA extraction kit such as the Qiagen QIAamp Circulating Nucleic Acid Kit as per the manufacturer's instructions (Qiagen, Hilden, Germany). Total nucleic acid is collected by such kits, and DNA is removed using a suitable kit such as the Qiagen RNase-free DNase Set or Ambion Turbo DNA-free Kit (Life Technologies, Carlsbad, Calif., USA). Eluted RNA, after DNA removal, is then detected as a quality control using a suitable total nucleic acid assessment technique such as the Agilent RNA pico Kit on Bioanalyzer equipment (Agilent Technologies, Eugene, Oreg., USA). RNA of sufficient quality is then stored for subsequent quantification.

### B. Reverse Transcription-PCR and Gene Sequencing

RNA (5-10 ng) may be reverse-transcribed using M-MLV Reverse Transcriptase (Invitrogen, Life Technologies, Inc.). Samples are amplified with PCR in a final reaction volume of 25 μl containing 2.5 μl of 10 times buffer, 0.1 μl of 10 mM dNTPs, 10 pmoles of each primer and 0.5 units of Taq DNA Polymerase. To confirm the presence and integrity of the cDNA template, the housekeeping gene, GAPDH, is amplified for each sample using primers GAPDH-5 (5′-ACCACAGTCCATGCCATCAC-3′; SEQ ID NO: 1) and GAPDH-3 (5′-TCCACCACCCTGTTGCTGTA-3′; SEQ ID NO: 2). Conditions may be as follows: an initial denaturation step for 5 minutes at 94° C., then 50 seconds at 94° C., 45 seconds at 55° C., and 1 min at 72° C. for 30 cycles, followed by an elongation step for 10 minutes at 72° C.

The cDNA obtained from the extracted total RNA may be analysed further, such as via a DNA microarray, in order to determine the identities and expression levels of genes expressed within the PBMCs and the tissue biopsy samples. Alternatively, reverse transcription and amplification can be performed using a suitable genome sequencing method, such as Ampliseq (Life Technologies, ThermoFisher, Austin, Tex.). Up to 20,000 genes can be sequenced and several libraries (one library per sample) can be analysed in one experiment. As an example, determination of the expression of the following cytochrome P450 mono-oxygenase genes linked to drug and xenobiotic compound metabolism may be determined in both the plasma as well as in the organ samples: CYP1A2; CYP1A1; CYP1B1; CYP2A6; CYP2A7, CYP2A13; CYP2B6; CYP2C8; CYP2C9; CYP2C18; CYP2C19; CYP2D6; CYP2E1; CYP3A4; CYP3A5 and CYP3A7. Other genes which may be determined include marker genes for hepatocytes, in order to determine the SCF for the particular individual (see Example 2, below).

The above protocol may be repeated as necessary for multiple individuals in order to generate data on expression levels of genes linked to drug and xenobiotic compound metabolism and/or the expression of organ marker genes. The data is suitable for interrogation via bioinformatics techniques to determine correlations between marker expression in circulating mRNA and expression of CYPs, for example, in the organ sample. The correlations are used to develop a virtual model of xenobiotic compound clearance that can be configured on a person by person basis in order to provide a virtual twin model of compound clearance within a given individual.

### Example 2

The following example provides a protocol for determining the degree of RNA shedding into circulation from hepatocytes in a particular subject, so establishing a robust and significant correlation function between hepatic protein levels and the corresponding plasma RNA concentrations.

Marker genes: A1BG (Alpha-1-B glycoprotein), AHSG (alpha-2-HS-glycoprotein), ALB (Albumin), APOA2 (Apolipoprotein A-II), C9 (Complement component 9), CFHR2 (Complement factor H-related 5), F2 (Coagulation factor II (thrombin)), F9 (Coagulation factor IX), HPX (Hemopexin), SPP2 (Secreted phosphoprotein 2), TF (Transferrin), MBL2 (mannose-binding lectin (protein C) 2); SERPINC1 (Serpin peptidase inhibitor, clade C (antithrombin), member 1) and FGB (Fibrinogen beta chain).

The use of an SCF based on the 13 selected genes reduces the effects of technical variability inherent to using only one gene, such as albumin (ALB), as a reference. It is known that the level of shedding in cancer patients can be higher (several fold) and can be more variable (several fold) than that in healthy controls. Without wishing to be bound by theory, the increased amount of RNA shedding (and observed larger variability) in cancer patients may result from cell death (necrosis), possibly also in response to chemotherapy. Nevertheless, the identification of this phenomenon permits correction and accommodation within models generated by the methods of the invention.

Therefore, due to the presence of different levels of RNA shedding amongst test subjects, the correction being applied to enzyme expression levels in plasma is as follows (e.g. using 13 markers).

$$SCF = {10}^{6} \cdot {\sum }_{i = 1}^{13} {[ cfRNA ]}_{{Marker}_{i}} / ( 13 \times {[ cfRNA ]}_{TOTAL} )$$

$${( {[ cfRNA ]}_{Enzyme} )}_{Normalized} = {[ cfRNA ]}_{Enzyme} / SCF$$

[cfRNA]_{TOTAL} is the total RNA reads in a library generated from the plasma sample of Experiment 1. The outcome should be a normalized reading for each enzyme expressed out of a million reads in a plasma sample of specified volume (1-5 ml).

### Example 3

### Quantification of Drug Metabolizing Enzymes in Liver from SCF Adjusted CYP cfRNA Levels Determined from Liquid Biopsy

The amounts of circulating plasma mRNA can be used to identify the relative abundance of a plurality of hepatic proteins that control xenobiotic compound clearance. Table 1 shows examples of abundance values that permit such estimation for four specific drug clearance enzymes in the liver of human subjects based upon the SCF-adjusted plasma concentration of the corresponding mRNA (i.e. [CYPnnn]ₚₗₐₛₘₐ]).

TABLE 1
Liver protein abundance equations from circulating RNA
measurements

| Enzyme | Equation |
|---|---|
| CYP3A4 | [CYP3A4]ₜᵢₛₛᵤₑ = 29.68 × [CYP3A4]ₚₗₐₛₘₐ + 0.62 |
| CYP2C9 | [CYP2C9]ₜᵢₛₛᵤₑ = 17.16 × [CYP2C9]ₚₗₐₛₘₐ + 0.46 |
| CYP1A2 | [CYP1A2]ₜᵢₛₛᵤₑ = 0.43 × [CYP1A2]ₚₗₐₛₘₐ + 0.18 |
| CYP2A6 | [CYP2A6]ₜᵢₛₛᵤₑ = 7.54 × [CYP2A6]ₚₗₐₛₘₐ + 0.54 |
| CYP2C19 | [CYP2C19]ₜᵢₛₛᵤₑ = 0.75 × [CYP2C19]ₚₗₐₛₘₐ − 0.06 |
| CYP2D6 | [CYP2D6]ₜᵢₛₛᵤₑ = 7.54 × [CYP2D6]ₚₗₐₛₘₐ − 0.29 |

As mentioned previously the abundance of the enzyme in the liver is capable of being correlated to a biological activity for that enzyme, thereby allowing for net intrinsic metabolic clearance to be determined.

The approach taken in the present invention allows for the creation of in silico models for individuals and populations that permit the more accurate predictive modelling of the speed of clearance of particular compounds, typically drugs or toxins. In the absence of the shedding correction the results are highly variable between individuals rendering liquid biopsy analysis impractical and inaccurate.

### Example 4

### A Cocktail Clearance Study to Inform and Develop an Improved PBPK Model for Drug Clearance in Healthy Adults

A study was designed that would enable generation of data on intrinsic clearance for a plurality of clearance proteins including CYPs and transporter proteins in a variety of tissues within human subjects. In the trial a plurality of healthy human volunteers are recruited for a 14 day clinical validation study. The volunteers are pre-screened to assess health status, medical history and to verify that they are non-smokers and are not receiving any medicines (prescription or OTC), nutritional or herbal supplements or recreational drugs. The candidates selected as subjects for the trial may include known fast, normal or slow metabolizers as determined by standard tests for CYP activity. In other trial designs particular patient cohorts may be selected based upon health status, ethnicity, sex, age, CYP genotype etc.

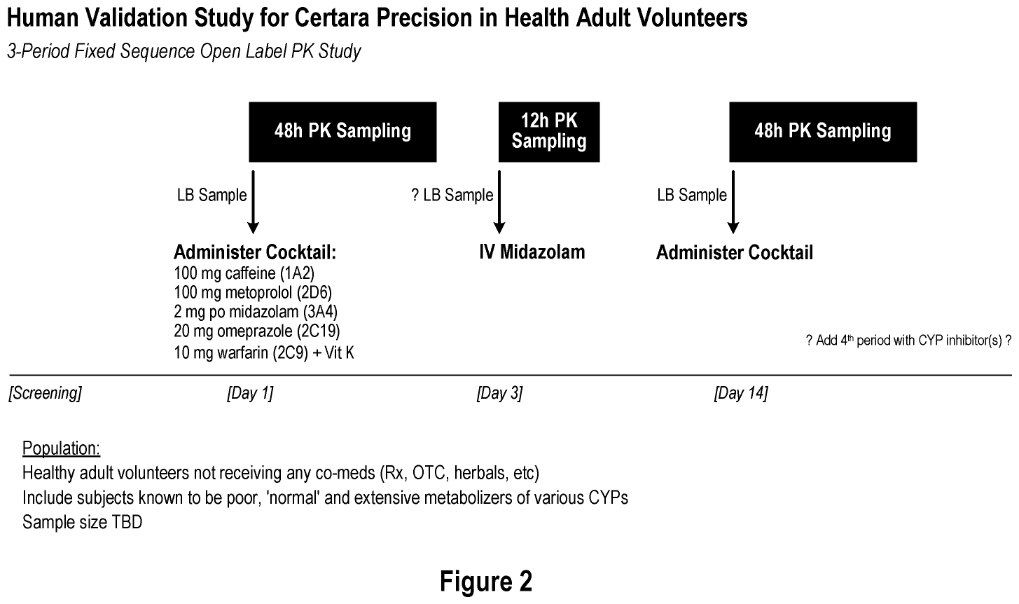

The design of the trial is set out in FIG. 2 in which liquid biopsies (blood and optionally urine) are taken during three sample windows following administration of a five drug cocktail. The five drugs are chosen in order to enable analysis of hepatic clearance from five different primary CYPs. The drugs are:

| | | | Clearance |
|---|---|---|---|
| | Dosage | Administration | protein |
| Compound | (mg) | route | (CYP) |
| Caffeine | 100 | oral | 1A2 |
| Metoprolol | 100 | oral | 2D6 |
| Midazolam | 2 | i.v. | 3A4 |
| Omeprazole | 20 | oral | 2C19 |
| Warfarin (+ vitamin K) | 10 | oral | 2C9 |

At least one liquid biopsy sampling occurs during a first window of 48 hours following administration of the cocktail. The concentration of circulating RNA for each of the identified clearance proteins in the biopsies is identified according to the methods set out above, particularly in Examples 1 to 3. In addition A second 12 hour window of liquid biopsy sampling may be implemented on day 3 post cocktail administration. It is an option to administer a further bolus of midazolam at this time point. A third 48 hour window occurs at day 14 post cocktail administration. At this point the liquid biopsies may be taken before the cocktail is administered again. In an optional extension to the trial design a further sampling window may be included in combination with administration of a selection of one or more inhibitors for the identified clearance proteins.

The data regarding hepatic clearance protein abundance within each subject over the time of the trial and in response to the administration of the various cocktail components can be analysed as described above. Data on clearance protein abundance and, thus, activity is able to be utilised in the development of improved PBPK models for the selected population. For example, the in vivo clearance protein abundance data may be combined with standard models for organ/tissue compartments to provide a refined model. For all individual subjects tested parameters such as the mean observed maximum concentration (Cₘₐₓ) as well as the area under the plasma concentration-time curve (AUC) for each drug used in the trial can be determined. The in vivo data can be compared to existing in silico simulations to see where matches or divergences exist. At all data points the liquid biopsy data can provide an estimate of the concentration/abundance of relevant xenobiotic clearance and transport proteins (e.g. CYPs and transporters) enabling a better fit to be made between the simulation and the observed in vivo data.

### Example 5

The liver has a central role in the metabolic elimination of not only endogenous compounds but also exogenous chemicals, including the majority of drugs. Variability in individual capacity for hepatic elimination of therapeutic drugs has been recognized and systematically studied over the past 50 years. Differences between patients in response to drug therapy are therefore common and they represent a persistent challenge for clinicians, often leading to sub-optimal patient care. Departure from the ‘one-size-fits-all’ approach to dosing, routinely practiced in the clinic, to more individualized patient care has been advocated by recent guidance, with the objective of improving drug effectiveness, reducing adverse reactions and decreasing medicines wastage. In most cases, implementation of precise dosing has been linked to differences in genetics, particularly those affecting drug pharmacology, such as the case of highly polymorphic cytochrome P450 enzymes CYP2D6 and CYP2C9. However, there is large variability in expression within each genotype, which can only be captured by expression data, and there are no known genetic signatures that define the wide variations observed in the expression of certain key drug-metabolizing enzymes, such as CYP3A4.

In this Example, the utility of the liquid biopsy test described in Examples 1 to 3 was demonstrated in relation to predicting patients at the extremes of drug exposure and guiding drug dose selection using an in silico model.

### Simulated Drug Exposure and Liquid Biopsy Guided Dose Adjustment using an in Silico Model

To simulate the impact of liquid biopsy input on dose selection, simulated drug trials were performed using Simcyp® 18 release 2 (Certara, Sheffield, UK). Three CYP3A substrates were evaluated: alprazolam (low clearance), midazolam (medium clearance) and ibrutinib (high clearance). Compound files were selected from the Simcyp® library. Exposure (expressed as AUC in μg h L⁻¹) to the three CYP3A substrates was assessed in simulated drug trials (details in Table 2), with dosing adjusted based on either stratified or individualized regimens determined using liquid biopsy measurement for the same virtual population (see Table 3). the same population was stratified into three groups based on their hepatic CYP3A4 content (liquid biopsy input). Simulations did not account for intestinal CYP3A4. For midazolam and ibrutinib, the top quartile (highest CYP3A4 levels) were administered a dose 2-fold higher than the standard dose; the bottom quartile (lowest CYP3A4 content) were administered a dose 3-fold lower. For alprazolam, a 2-fold dose increase for the top quartile and a 2-fold dose reduction for the bottom quartile were made. The dose selection for the two quartiles was guided by expression levels and the outcome of the uniform dosing simulation. The middle group (the remaining 50% of the population) were administered a standard dose. Individualized dosing was performed for the same virtual group based on individual dosage adjustment using patient-specific ratios of hepatic CYP3A4 content, simulating an individual liquid biopsy result, relative to the population average.

The dose adjustment ratios in all cases were informed by simulated liquid biopsy measurements. The outcome was compared to a uniform clinical dose.

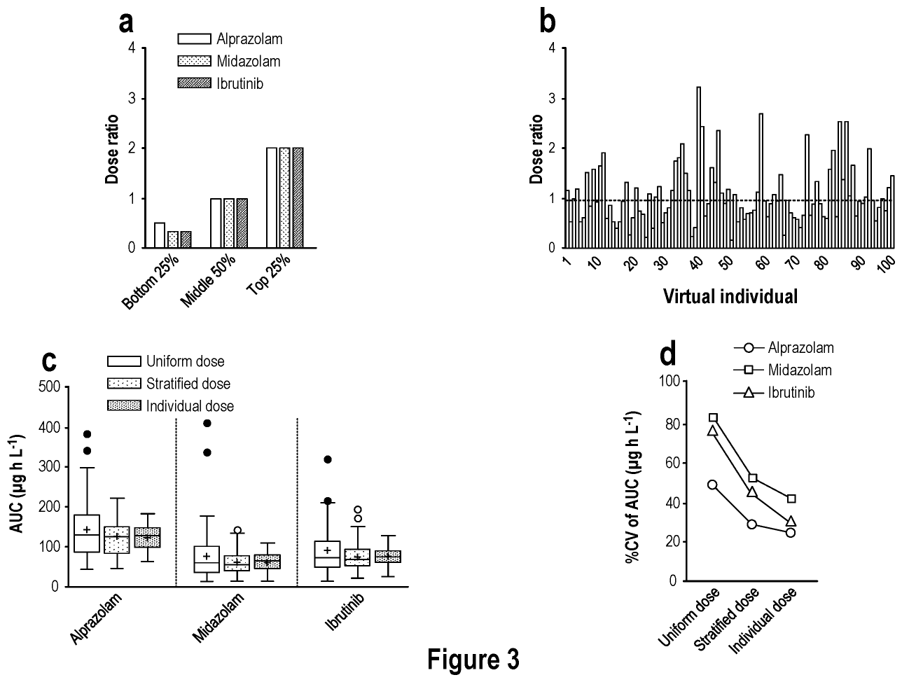

The simulations demonstrated a similar level of drug exposure as AUC but with a significant reduction in variability (% CV) with stratified and individualized dosing compared to uniform dosing for all three drugs (FIG. 3c and d). Using stratified dosing, the reduction in variability was approximately 1.7-fold compared to conventional uniform dosing. Individualized dosing resulted in a further reduction of variability in exposure from 50% to 25% (2 fold), 84% to 42% (2 fold) or 76% to 31% (2.5 fold), following an oral dose of alprazolam, midazolam or ibrutinib, respectively (FIG. 3d). The reduced variability in simulated exposure after an oral dose of the three substrates suggests a significantly more precise oral dose selection can be made for individual patients using tools, such as models, informed by the liquid biopsy test.

Applications of the presently described approaches include patient stratification to achieve more precise dosing and improved characterization of volunteers prior to enrolment in clinical trials. The requirement for changing a dosage regimen in these applications is highlighted in cases of adverse drug reactions or when a recommended dose is ineffective. Toxicity cases are associated with high levels of exposure to the drug, whereas cases of ineffective dosing are characterized by low exposure. We assessed the developed test as a tool to identify patients at the extremes of drug exposure (top and bottom quartiles). Accurate predictions were achieved for both groups, especially in the case of P450 enzymes. Taking the main drug-metabolizing enzyme, CYP3A4, as an example, the test identified the top quartile at 99% confidence and the bottom quartile at 84% confidence. Simulated trials of three CYP3A substrates (alprazolam, midazolam and ibrutinib) demonstrated reduced variability in exposure (by 2-2.5 fold) following oral dose adjustment using liquid biopsy input. The reduced variability in predicted drug exposure, that is apparent from FIG. 3d at least, demonstrates that far more precise dose selection for a given patient can be made in advance of initiating therapy using the approaches of the present invention as described herein.

In conclusion, this Example demonstrates quantitative liquid biopsy can inform PBPK modelling based on plasma measurements to determine accurate liver content of key enzymes and transporters. The use of this approach facilitates the deployment of precise and effective dosage regimens (precision dosing), as an essential element of precision medicine.

TABLE 2
Demographic and clinical detaisl of the virtual population (n = 100) used for a simulated drug trial.

| | | | | | | | | CYP3A4 | | | |
|---|---|---|---|---|---|---|---|---|---|---|---|
| | | | | | Serum | | Haema- | abundace | liver | Liver | Enzyme |
| | Age | Weight | Height | | Creatinine | HSA | tocrit | (pmol mg⁻¹ | weight | protein | content |
| | (years) | (kg) | (cm) | Sex | (μmol L⁻¹) | (g L⁻¹) | (%) | protein) | (g) | (mg) | (μmol) |
| Mean | 30.04 ± | 81.31 ± | 176.16 ± | M | 77.51 ± | 47.89 ± | 42.68 ± | 140.20 ± | 1747.71 ± | 38.86 ± | 9.51 ± |
| | 7.37 | 13.67 | 6.42 | | 11.90 | 5.00 | 2.55 | 62.33 | 248.88 | 10.38 | 5.20 |
| CV (%) | 24.53 | 16.82 | 3.65 | | 15.35 | 10.45 | 5.98 | 44.46 | 14.24 | 26.72 | 54.74 |
| Median | 27.36 | 79.81 | 176.14 | | 76.23 | 47.20 | 42.55 | 127.76 | 1784.21 | 37.36 | 8.32 |
| Range | 20.54- | 52.74- | 156.83- | | 54.71- | 37.55- | 37.08- | 33.86- | 1155.22- | 18.69- | 1.5928.45 |
| | 49.64 | 119.49 | 188.78 | | 108.88 | 62.67 | 49.17 | 357.94 | 2353.99 | 73.10 | |

TABLE 3
The dosing procedure for the three simulations: uniform dosing, stratified
dosing and individualized dosing of alprazolam, midazolam and ibrutinib.
Dose adjustment calculation for the stratified and individualized dose was
based on expression data (liquid biopsy input) relative to population average
(population of 1000 individuals). Oral route of administration was used for
simulations.

| | Uniform dosing | Stratified dosing | | Individualized dosing |
|---|---|---|---|---|
| Alprazolam | Dose 0.5 mg | Bottom 25%; | Dose 0.25 mg | Mean dose: 0.55 ± 0.30 mg |
| | | Middle 50%; | Dose 0.5 mg | Dose range: 0.09-1.64 mg |
| | | Top 25%; | Dose 1 mg | |
| Midazolam | Dose 5 mg | Bottom 25%; | Dose 1.67 mg | Mean dose: 5.5 ± 2.99 mg |
| | | Middle 50%; | Dose 5 mg | Dose range: 0.92-16.38 mg |
| | | Top 25%; | Dose 10 mg | |
| Ibrutinib | Dose 140 mg | Bottom 25%; | 46.67 mg | Mean dose: 153.31 ± 83.92 mg |
| | | Middle 50%; | 140 mg | Dose range: 25.65-458.76 mg |
| | | Top 25%; | 280 mg | |

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1. A process for establishing a virtual physiologically based pharmacokinetic (PBPK) model in a population comprised of a plurality of individual subjects that has been or may be exposed to a xenobiotic molecule, the process comprising the steps of:
a) isolating total cell free RNA (cfRNA_{TOTAL}) from a liquid biopsy obtained from each individual subject comprised within the population;
b) quantifying an amount of a first cell free RNA (cfRNA) present in the liquid biopsy, wherein the first cfRNA originates from a specified organ/tissue within the bodies of the subjects, and wherein the first cfRNA encodes a protein from the organ/tissue that is involved in pharmacokinetic activity relevant to the xenobiotic molecule selected from one or more of the group consisting of: absorption; distribution; localization; biotransformation; and excretion of the xenobiotic molecule;
c) performing an adjustment function on the amount of the first cfRNA so as to correct for inherent levels of RNA shedding within each of the plurality of individual subjects;
d) identifying the abundance of the protein within the specified organ/tissue for each subject by comparison of the corrected amount of the first cfRNA with abundance data for the corresponding amount of protein in the specified organ/tissue;
e) determining a pharmacokinetic activity relevant to the xenobiotic molecule for each individual subject based upon the abundance of the protein within the specified organ/tissue of the subject;
f) combining the pharmacokinetic activities of each individual subject to create a data set of pharmacokinetic activities for the population of individuals; and
g) utilising the data set to generate the PBPK model.

2. A process for establishing a personalised PBPK model for an individual subject that has been or may be exposed to a xenobiotic molecule, the process comprising the steps of:
i isolating total cell free RNA (cfRNA_{TOTAL}) from a liquid biopsy obtained from the individual subject;
ii quantifying an amount of a first cell free RNA (cfRNA) present in the liquid biopsy, wherein the first cfRNA originates from a specified organ/tissue within the body of the subject that is involved in pharmacokinetic activity relevant to the xenobiotic molecule selected from one or more of the group consisting of: absorption; distribution; localization; biotransformation; and excretion of the xenobiotic molecule;
iii performing an adjustment function on the amount of the first cfRNA so as to correct for inherent levels of RNA shedding in the individual subject;
iv identifying the abundance of the protein within the organ/tissue of the subject by comparison of the corrected amount of the first cfRNA with abundance data for the corresponding amount of protein in the organ/tissue;
v determining a pharmacokinetic activity relevant to the xenobiotic molecule for the individual subject based upon the abundance of the protein within the organ/tissue of the subject; and
vi generating the virtual PBPK model for the individual subject.

3. The process of claim 1, wherein the adjustment function comprises identifying the amount of the first cfRNA present by correcting against a RNA organ Shedding Correction Factor (SCF) that is determined for the individual subject by performing an analysis of the cfRNA_{TOTAL} in order to quantify an amount of mRNA present within the cfRNA_{TOTAL} that corresponds to each of two or more marker genes, wherein a marker gene is defined as a gene that is expressed principally and consistently in the organ/tissue; and determining SCF as the mean concentration of mRNA of the each of two or more marker genes present within the cfRNA_{TOTAL}.

4. The process of claim 3, wherein the SCF is determined for the subject by isolating cfRNA_{TOTAL} from a liquid biopsy obtained from an individual subject, performing an analysis of the cfRNA_{TOTAL} in order to quantify an amount of two or more marker genes mRNAs present, designated as [cfRNA]_{Marker}, wherein a marker gene is defined as a gene that is expressed principally and consistently in the organ/tissue and at a high level; and determining the SCF according to the formula A:
SCF=10⁶·Σᵢ₌₁^{N}[cfRNA]_{Marker}_{ᵢ}/(N×[cfRNA]_{TOTAL}) A
where N is equal to the number of marker genes quantified.

5. The process of claim 4, wherein N is at least three, suitably at least five, typically at least eight and optionally at least ten.

6. The process of claim 1, wherein the organ/tissue is selected from one or more of the group consisting of: the liver; the kidney; the gastrointestinal tract; the brain/CNS; and the pancreas.

7. The process of claim 1, wherein the process comprises quantifying an amount of a second cell free RNA (cfRNA) present in the liquid biopsy, wherein the second cfRNA originates from an organ/tissue within the body of the subject which has the capacity to undertake metabolic xenobiotic clearance, and wherein the second cfRNA encodes a protein from the organ/tissue that is involved in metabolic xenobiotic clearance.

8. The process of claim 1, wherein the process comprises quantifying an amount of a plurality of cell free RNAs (cfRNAs) present in the liquid biopsy, wherein the each of the plurality of cfRNAs originates from an organ/tissue within the body of the subject which has the capacity to undertake metabolic xenobiotic clearance, and wherein the plurality cfRNAs encode proteins from the organ/tissue that are involved in metabolic xenobiotic clearance.

9. The process of claim 1, wherein the organ/tissue-derived cfRNA encodes a xenobiotic handling protein selected from the group consisting of: a xenobiotic clearance protein; a xenobiotic metabolising enzyme; and a xenobiotic transporting protein.

10. The process of claim 9, wherein the cfRNA encodes a cytochrome P450 monooxygenase (CYP) protein.

11. The process of claim 10, wherein the CYP is selected from one of the group consisting of: CYP1A1; CYP1A2; CYP1 B1; CYP2A6; CYP2A7; CYP2A13; CYP2B6; CYP2C8; CYP2C9; CYP2C18; CYP2C19; CYP2D6; CYP2E1; CYP3A4; CYP3A5; and CYP3A7.

12. The process of claim 9, wherein cfRNA encodes a transferase selected from one of the group consisting of: a methyltransferase; a sulfotransferase; an N-acetyltransferase; a glucuronosyltransferase including, but not limited to, one or more of the group consisting of UGT1A1, UGT1A3, UGT1A4, UGT1A6, UGT1A9, UGT2B4, UGT2B7, UGT2B15 and UGT2B17; a glutathione-S-transferase; and a choline acetyl transferase.

13. The process of claim 9, wherein the cfRNA encodes a transporting protein selected from an ATP-binding cassette (ABC) transporter or a solute carrier (SLC) transporter.

14. The process of claim 1, wherein the liquid biopsy comprises a sample of a bodily fluid selected from one of the group consisting of: blood; urine; saliva; semen; tears; lymphatic fluid; stool; bile; cerebrospinal fluid; and a mucus secretion.

15. The process of claim 14, wherein the liquid biopsy comprises whole blood, or a component thereof selected from serum or plasma.

16. The process of claim 1, wherein the xenobiotic is a pharmaceutical compound or a drug.

17. The process of claim 1, wherein the xenobiotic is a toxin or an environmental contaminant.

18. The process of claim 1, wherein the individual subject is a human.

19. The process of claim 1, wherein the individual subject is a non-human animal.

20. A method of treating an individual subject, wherein the individual is the intended recipient of a pharmaceutical treatment, the method comprising establishing a personalised virtual PBPK model of the body of the individual subject prior to or during treatment, the process comprising the steps of:
isolating total cell free RNA (cfRNA_{TOTAL}) from a liquid biopsy obtained from the individual subject;
quantifying an amount of a first cell free RNA (cfRNA) present in the liquid biopsy, wherein the first cfRNA originates from a specified organ/tissue within the body of the subject, and wherein the first cfRNA encodes a protein from the organ/tissue that is involved in pharmacokinetic activity relevant to the pharmaceutical compound selected from one or more of the group consisting of: absorption; distribution; localization; biotransformation; and excretion of the pharmaceutical compound;
performing an adjustment function on the amount of the first cfRNA so as to correct for inherent levels of RNA shedding in the individual subject;
identifying the abundance of the protein within the organ/tissue of the subject by comparison of the corrected amount of the first cfRNA with abundance data for the corresponding amount of protein in the specified organ/tissue of the subject;
determining a pharmacokinetic activity relevant to the pharmaceutical compound for the individual subject based upon the abundance of the protein within the specified organ/tissue of the subject;
generating the personalised virtual PBPK model of pharmaceutical compound clearance for the individual subject; and
treating the individual according to a dosage regimen for the pharmaceutical compound that is optimized to the individual based upon their personalised virtual PBPK model.

21. The method of claim 20, wherein the individual subject is a human.

22. The method of claim 20, wherein the individual subject is a non-human animal.

23. The method of claim 20, wherein the individual subject is suffering from a disease selected from any one of the group selected from: cancer; inflammatory disease; auto-immune disorders; allergy; metabolic diseases, including metabolic deficiency; degenerative diseases, including neurodegenerative diseases (e.g. Alzheimer's, Parkinson's, ALS, multiple sclerosis, Huntington's); psychiatric disorders; and infection, including chronic or acute infection from bacterial, viral, fungal or parasitic pathogens.


SEQUENCE LISTING (2 sequences)

SEQ ID NO: 1 - DNA, 20 bp, Homo sapiens
  misc_feature (1)..(20): GADPH PRIMER
accacagtccatgccatcac

SEQ ID NO: 2 - DNA, 20 bp, Homo sapiens
  misc_feature (1)..(20): GADPH PRIMER
tccaccaccctgttgctgta